SEQUENCE LISTING (3 sequences)

SEQ ID NO: 1 - protein, 16 aa, Artificial Sequence
  Description of Artificial Sequence Synthetic peptide
MHHHHHHGSENLYFQG

SEQ ID NO: 2 - protein, 9 aa, Artificial Sequence
  Description of Artificial Sequence Synthetic peptide
MHHHHHHGS

SEQ ID NO: 3 - protein, 6 aa, Artificial Sequence
  Description of Artificial Sequence Synthetic 6xHis tag
HHHHHH

(19) United States
(12) Patent Application Publication
(10) Pub. No.: US 2024/0228730 A1
(43) Pub. Date: Jul. 11, 2024

(54) Methods and Systems for Single-Step Decontamination and Enzymatic Degradation of Bio-Based Polymers

(71) Applicant: Kimberly-Clark Worldwide, Inc., Neenah, WI (US)

(72) Inventors: Stephen Quirk, Alpharetta, GA (US); David W. Koenig, Menasha, WI (US)

(21) Appl. No.: 18/558,892

(22) PCT Filed: May 24, 2022
(86) PCT No.: PCT/US2022/030717

§ 371 Date: Nov. 3, 2023

Related U.S. Application Data
(60) Provisional application No. 63/194,487, filed on May 28, 2021.

(51) Int. Cl. C08J 11/10; A61L 11/00; C12N 9/18

(52) U.S. Cl. CPC C08J 11/105; A61L 11/00; C12N 9/18; C08J 2367/04

(57) ABSTRACT

Methods and systems for simultaneous degradation and decontamination of biopolymers such as polyhydroxyalkanoates are described. The enzymes encompass extremophilic enzymes that can be incorporated into a biodegradation process carried out at an environmental condition that is detrimental to a mesophilic pathogen. The materials, methods, and systems of the present disclosure are particularly directed to degrading and decontaminating used personal care products containing polyhydroxyalkanoate polymers using extremophilic and/or polyextremophilic depolymerase enzymes.

### CROSS-REFERENCE TO RELATED APPLICATION

This application claims filing benefit of U.S. Provisional Patent Application Ser. No. 63/194,487 having a filing date of May 28, 2021, which is incorporated herein by reference for all purposes.

### SEQUENCE LISTING

This application contains a Sequence Listing which has been submitted electronically in ASCII format and is hereby incorporated by reference in its entirety. Said ASCII copy, created on May 17, 2022, is named KCX-2023-PCT_Sequence List.txt and is 1,051 bytes in size.

### BACKGROUND

It has been estimated that over 300,000,000 metric tons of petroleum-based polymers are being produced each year with global production continuing to increase. A significant portion of these polymers are used to produce single-use products, such as plastic drinking bottles, straws, packaging, and personal care products. Most of these plastic products are discarded and do not enter the recycle stream. As the worldwide single-use plastic epidemic worsens, it becomes paramount to identify fully renewable plastics and develop methods and materials that provide for industrial processing of renewable plastics.

Biodegradable polymers produced from renewable resources (also termed “biopolymers”) hold great promise for reducing the global accumulation of petroleum-based plastics in the environment. One such class of biopolymers are the polyhydroxyalkanoates (PHA). Much work has been accomplished on the PHA family, most notably the polyhydroxybutyrate (PHB) polymers including poly-3-hydroxybutyrate (P3HB), poly-4-hydroxybutyrate (P4HB), polyhydroxyvalerate (PHV), polyhydroxyhexanoate (PHH), polyhydroxyoctanoate (PHO) and their copolymers. Of particular advantage, PHA exhibit thermoplastic properties that are very similar to some petroleum-based polymers and thus represent viable replacements for petroleum-based polymers such as polypropylene and polyethylene.

PHA are naturally produced across many bacterial, fungal, and archaeal lineages including Azotobacter, Ralstonia, Burkholderia, Protomonas, Bacillus, and Schlegelella for use as an energy sink. Production of PHA polymers involves a three-step enzymatic mechanism that begins with acetyl coenzyme A. In forming PHB, the first step is catalysis of acetyl-CoA by PhaA (a β-ketothiolase) to form β-ketoacyl-CoA. This in turn is converted in a NADP-dependent reaction into R-3-hydroxyacyl-CoA by the PhaB enzyme (a β-ketoacyl-CoA reductase). The final step, catalyzed by PhaC (a PHB synthase), is the polymerization of R-3-hydroxyacyl-CoA into PHB.

In nature, to retrieve the energy stored in the polymer, biodegradation is accomplished by a PHA depolymerase (PHADase). Unfortunately, post-consumer products are often contaminated with waste materials such as food waste and human waste that encourage proliferation of a myriad of pathogenic organisms including viruses, bacteria, fungi, parasites, protozoans, etc. As a result, successful biodegradation of post-consumer products requires both decontamination to inactivate pathogens and degradation of the polymers. Typically, either the contaminated portions of a product are separated from other uncontaminated portions prior to degradation treatments, or decontamination and biodegradation are carried out on the entire product, but in separate operations, both of which add to processing costs.

A need exists for systems and methods that can increase the use of biopolymers in consumer products. Systems and methods that can provide simultaneous decontamination and biodegradation of post-consumer products, for instance post-consumer personal care products, would be of great benefit in the art.

### SUMMARY

In general, the present disclosure is directed to methods and systems for simultaneous degradation and decontamination of PHA polymers. PHA polymers for degradation can be components of post-consumer products, such as post-consumer personal care products or food industry products, which may be contaminated with one or more mesophilic pathogens. Currently, a significant portion of post-consumer products including, without limitation, packaging, straws, cups, bottles, shopping bags, eating utensils, trays, and personal care products such as personal care garments (e.g., diapers, child training pants, disposable swim pants, feminine hygiene products, adult incontinence products), tampon dispensers, medical supplies, etc., are made from petroleum-based polymers. Significant efforts are currently underway to incorporate biopolymers such as PHA into such products as well as improve and encourage the recycling of the biopolymers. The present disclosure is directed to improved methods and systems that can be used for simultaneous decontamination and biodegradation of biopolymers in small or large settings.

In one aspect, disclosed are methods for treating post-consumer products that include a PHA. For instance, a method can include contacting a post-consumer product, e.g., a post-consumer personal care product, a product used in the food industry, etc., with an extremophilic PHA depolymerase (PHADase) and/or a microorganism that expresses the extremophilic PHADase. The microorganism can be an extremophile that naturally exists at conditions including one or more of extreme temperature, pressure, salinity, acidity, alkalinity, radiation, etc., and the extremophilic PHADase can exhibit activity at such conditions. The PHADase can degrade a PHA of the post-consumer products. The contact can take place at an environmental condition that is deleterious to mesophilic pathogens and at which the extremophilic PHADase is active, e.g., one or more of extreme temperature, pressure, salinity, acidity, alkalinity, radiation, etc. In one embodiment, an extremophilic PHADase can be polyextremophilic, i.e., produced from a microorganism that naturally exists in an environment that includes multiple extreme environmental conditions. In one embodiment, the method can include contacting a post-consumer product with multiple extremophilic PHADase, one or more of which can be a polyextremophilic PHADase.

In one aspect, disclosed is a system that can be utilized for simultaneous decontamination and biodegradation of a post-consumer product. For instance, a system can include a reaction chamber within which a PHA-containing post-consumer product can contact an enzyme, an environmental control system configured to maintain one or more extreme environmental conditions within the contact chamber for a period of time, and an extremophilic PHADase and/or a microorganism that expresses an extremophilic PHADase. In various aspects, a system can be small, for instance designed for a single household or place of business, or larger, for instance designed for use in a neighborhood or community recycling center, or larger yet, for instance designed for use on an industrial scale.

### BRIEF DESCRIPTION OF THE DRAWINGS

A full and enabling disclosure of the present subject matter, including the best mode thereof to one of ordinary skill in the art, is set forth more particularly in the remainder of the specification, including reference to the accompanying figures in which:

FIG. 1 schematically illustrates a bioreactor system as may be utilized according to disclosed methods.

FIG. 2 schematically illustrates a smaller bioreactor system as may be utilized according to disclosed methods.

FIG. 3 illustrates enzymatic activity and colony forming units as a function of time for a PHBDase from the mesophilic Pseudomonas geniculate.

FIG. 4 illustrates enzymatic activity and colony forming units as a function of time for a PHBDase from the thermophilic Lihuaxuella thermophila.

FIG. 5 illustrates enzymatic activity and colony forming units as a function of time for a PHBDase from the thermophilic Schtegelella sp. TD0723.

FIG. 6 illustrates enzymatic activity and colony forming units as a function of time for a PHBDase from the halophilic Halomonas aquamarina.

FIG. 7 illustrates enzymatic activity and colony forming units as a function of time for a PHBDase from the halophilic Halomarina oriensis.

FIG. 8 illustrates enzymatic activity and colony forming units as a function of time for a PHBDase from the acidophilic Acidiphilium cryptum.

FIG. 9 illustrates enzymatic activity and colony forming units as a function of time for a PHBDase from the acidophilic Alkalimonas amylolytica.

FIG. 10 illustrates the depolymerization of PHB into hydroxybutyrate by four polyextremophile PHBases.

FIG. 11 illustrates the survival of E. coli as a function of time under individual and combined reaction conditions favorable for a PHBase of a polyextremophile Thermobifida halotolerans.

FIG. 12 illustrates the survival of E. coli as a function of time under individual and combined reaction conditions favorable for a PHBase of a polyextremophile Georgenia satyanarayanai.

FIG. 13 illustrates the survival of E. coli as a function of time under individual and combined reaction conditions favorable for a PHBase of a polyextremophile Natronococcus sp. LS1_42.

FIG. 14 illustrates the survival of E. coli as a function of time under individual and combined reaction conditions favorable for a PHBase of a polyextremophile Marinobacter profundi.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In order to reduce and eliminate polymer waste, not only is it necessary to replace petroleum-based polymers with biopolymers, but improved post-consumer processing of these polymers is also required. Significant research is currently underway to improve mass processing of biopolymers. Such polymers are well suited to producing all different types of single-use products, such as drink bottles, containers, packaging, and the like. In addition, those skilled in the art have proposed replacing petroleum-based polymers found in disposable personal care products such as incontinence products with biopolymers, such as PHA polymers. The use of biopolymers to replace petroleum-based polymers will make significant strides in creating a sustainable economy.

Most plastic single-use products, such as packaging, straws, cups, bottles, shopping bags, eating utensils, trays, personal care products, etc. are buried in landfills after use. Even if made from biopolymers, these materials will still require a significant amount of time to degrade and often are combined with other, less degradable materials, which can further slow natural degradation of the biopolymers. Moreover, single-use products such as food packaging or utensils and personal care products can be contaminated with food or human waste, e.g., feces, urine, blood, menstrual fluid, food remains, etc. at disposal and such waste can carry pathogenic contamination. In order to further improve the sustainability equation, the present disclosure is directed to a method and system for simultaneous decontamination of post-consumer products and degradation of biopolymers contained in the post-consumer products, and in one particular embodiment, simultaneous decontamination and degradation of post-consumer personal care products.

In this regard, the present disclosure is generally directed to systems and methods that utilize extremophilic enzymes (also referred to as extremozymes) at an environmental condition at which the extremophilic enzyme is active but that is deleterious for mesophilic pathogens. By use of disclosed methods and systems, simultaneous decontamination of post-consumer products and degradation of biopolymers contained in the products can be attained. As such, disclosed methods and systems can provide a route to a more sustainable economy while reducing risk of pathogens that may be present in post-consumer products.

Eliminating pathogenic contaminants can reduce risk to process operators as well as reduce the risk of process equipment contamination. Moreover, as disclosed methods are carried out at an environmental condition that is deleterious to mesophilic pathogens, the systems can be utilized and the methods carried out without additional sterilization procedures necessary for treatment of the pre-processed waste, the bioreactors, or the final post-processing reaction mixture. This can simplify an overall treatment process and can reduce costs, for instance as the post-processing mixture can be safely discarded without additional processing. In addition, disclosed methods provide a route for improving biodegradation of post-consumer products in non-industrial settings, such as in a home or small business by a consumer, or in a small neighborhood or community recycling center. Disclosed methods and systems can also be used without the need to include additional antipathogenic agents, e.g., antibacterial processing aids, and as such can help to prevent overuse of such agents and associated development of antibiotic resistance in pathogens.

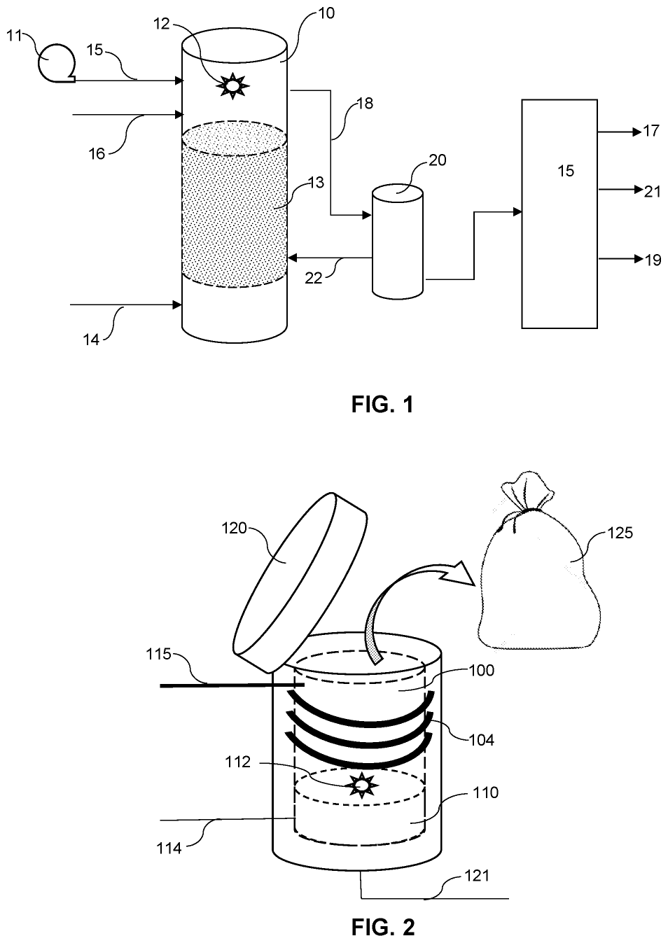

A system as disclosed herein can incorporate a bioreactor within which simultaneous decontamination and depolymerization operations can be carried out. One embodiment of a bioreactor system is schematically illustrated in FIG. 1. Another example of a bioreactor system, for instance as may be utilized in a smaller application, such as in a household or small business, is schematically illustrated in FIG. 2.

Whether in a large or small application, a bioreactor can generally include a reaction chamber 10, 100 that can be formed of a material that can contain the enzymes, reactants, and products at the desired reaction conditions. For instance, a bioreactor can include stainless steel, borosilicate glass, Teflon® and other nonreactive temperature insensitive composite polymers, and so forth. A reaction chamber 10, 100 can provide a contact area between an extremozyme and a post-consumer product for a period of time to encourage degradation of a biopolymer contained in the post-consumer product, with the contact taking place and at an environmental condition at which the extremophilic enzyme is active and at which a mesophilic pathogen can be rendered non-pathogenic.

Mesophilic pathogens that can be rendered non-pathogenic by disclosed methods and systems can include, without limitation, viruses, bacteria, fungi, and protozoans. As utilized herein, the terms “mesophile” and “mesophilic” refer to organisms that naturally exist in environmental conditions at which humans generally co-exist with the organism, including near human body temperature (e.g., from about 20° C. to about 45°), a saline content in water of from about 5 to about 18 parts per thousand (also referred to as mesohaline), about one atmosphere pressure (e.g., from about 20 kPa to about 110 kPa), and near neutral pH (e.g. from about pH 5 to about pH 8.5, also referred to as neutrophiles or neutrophilic). Typical bacterial pathogens encompassed herein can include those commonly found in human stool such as, and without limitation to, those of a genus Streptococcus, Bifidobacterum, Lactobacillus, Staphylococcus, Clostridium, Enterobacteriaceae, or Bacteroides.

To encourage the desired biodegradation activity, an extremophilic enzyme and/or a microorganism that expresses an extremophilic enzyme can be located in a reaction chamber 10, 100 in conjunction with a post-consumer product. For instance, an extremozyme and/or a microorganism that expresses an extremozyme can be fed to a reaction chamber 10 via an inlet stream 14, can be previously retained within a bed 13 within the reaction chamber 10, or any combination thereof. When considering a smaller system such as for household use, an extremozyme and/or a microorganism that expresses an extremozyme can in one embodiment be fed to the reaction chamber 100 by the user, for instance from a separately provided packet or container that carries the enzyme until use.

In general, any suitable extremozyme capable of degrading a biopolymer, particularly a polyhydroxyalkanoate polymer, at one or more environmental conditions that are deleterious to a mesophilic pathogen is encompassed herein. For instance, an extremophilic enzyme can be one that is naturally expressed from an extremophilic bacteria or archaea and that is active at an environmental condition suitable for decontamination of a mesophilic pathogen. Extremophiles encompassed herein can include, without limitation, thermophiles, cryophiles, acidophiles, alkaliphiles, anaerobes, capnophiles, halophiles, piezophiles, and radioresistant organisms. As utilized herein the term “active” with regard to an extremophilic enzyme generally refers to the ability of the enzyme to catalyze a reaction at a reaction rate of 10% or greater of the maximum rate of the system (Vₘₐₓ) given a suitable substrate concentration. In one particular embodiment, the enzyme can be an extremophilic poly[R-3-hydroxybutyrate] depolymerase (PHBDase) that degrades a poly[R-3-hydroxybutyrate] (PHB) according to the following reaction:

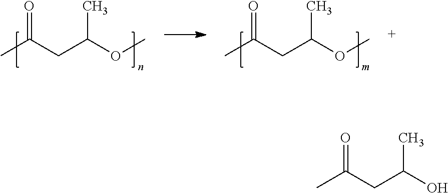

wherein m<<<n and represents small oligomers, e.g., primarily 2-4 mers.

In some embodiments, an extremophilic enzyme can include a thermophilic enzyme for which the optimum temperature (Tₒₚₜ, that temperature at which a maximum reaction rate can be achieved given suitable substrate) can be above that at which a mesophilic enzyme can survive. For instance, an enteric vegetative bacteria, fungi, and protozoa will be inactivated if exposed to a temperature of about 6000 for a period of time of about 30 min or more. Enveloped viruses can be inactivated at a similar temperature, e.g., about 60° C. or greater, with some non-enveloped viruses requiring higher temperatures, e.g., about 80° C. or greater. Overall most enteric viruses encountered in the stool can be inactivated upon exposure to a temperature of about 80° C. for about 6 min. Bacterial spores can require high temperatures for longer exposure times, such as a temperature of about 9000 or greater for an exposure time of about 1 hour. Escherichia coli, Klebsiella pneumoniae, Serratia marcescens, Pseudomonas aeruginosa, and Acinetobacter calcoaceticus can all be killed upon exposure to a temperature of from about 60° C. to about 70° C. for about 30 min.

Accordingly, in some embodiments, an extremophilic enzyme for use in disclosed methods and systems can be a thermophilic enzyme that exhibits a Tₒₚₜ of about 40° C. or greater, about 5000 or greater, about 6000 or greater, about 7000 or greater, about 8000 or greater, or about 9000 or greater in some embodiments. Exemplary thermophiles (and thermophilic enzymes produced thereby) encompassed herein can include, without limitation, Alicyclobacillus pomorum (WP-084453829), Amycolatopsis thermoflava (WP-123687648), Amycolatopsis thermalba (WP-094002797), Amycolatopsis rumanii (WP-116109633), Azospirillum thermophilum (WP-109324320), Deinococcus actinosclerus (WP-082689076), Fervidobacterium gondwanense (SHN54810), Gandjariella thermophila (WP-137812779), Georgenia satyanarayanai (WP-146237554), Hyphomanas sp. (HA037884), Lihuaxuella thermophila (WP-089972404), Microbulbifer thermotolerans (P-197462976), Minwuia thermotolerans (WP-206420073), Rhodopseudomonas thermotolerans (WP-114356866), Rhodopseudomonas pentothenatexigens, (WP-114356866), Streptomyces thermovulgaris (WP-067396676), Thermanaeromonas toyohensis (WP-084666479), Thermoactinomyces sp. CICC 10523 (WP-198056464), Thermoactinomyces daqus (WP-033100012), Thermoactinospora sp. (NUT44302), Thermoactinospora rubra (WP-084965756), Thermobifida halotolerans (WP-068692693), Thermobifida fusca (WP-011290529), Thermobispora bispora (WP-206206594), Thermocatellispora tengchongensis, (WP-185055796), Thermochromatium tepidum (WP-153975900), Thermocrispum municipal (WP-028851041), Thermoflavimicrobium dichotomicum (WP-093229000), Thermogemmatispora carboxidivorans (WP-081839208), Thermogemmatispora aurantia (WP-151728970), Thermogemmatispora tikiterensis (WP-11243376), Thermogemmatispora onikobensis (WP-084659191), Thermoleophilaceae bacterium (MBA2429278), Thermomonospora echinospora (WP-160147065), Thermomonospora cellulosilytica (WP-182704610), Thermomonospora amylolytica (WP-198679325), Thermostaphylospora chromogena (WP-093263254), Thermus thermophilus (WP-197735236), Thermus aquaticus (WP-053768217), Thermus islandicus (HE042284).

Temperature-based extremozymes encompassed herein are not limited to high temperature thermophilic enzymes, however, and low temperature cryophilic enzymes (also referred to a psychrophilic enzymes) can be utilized in some embodiments. For instance, many bacterial strains will fail to multiply, but will still survive upon exposure to a temperature of about 10° C. for a period of time of about 6 hours. Thus, in some embodiments, a cryophilic enzyme capable of activity at a temperature of about 10° C. or less, for instance 7° C. or less, or from about −15° C. to about 10° C. in some embodiments, can be utilized. Exemplary psychrophiles (and psychrophilic enzymes produced thereby) encompassed herein can include, without limitation, Alteromonas oceani (WP-123325050), Alteromonas alba (WP-105936495), Alteromonas sp. 38 (WP-201299304), Alteromonas macleodii (WP-156078157), Alteromonas ponticola (WP-169211550), Alteromonas lipolytica (WP-070178363), Arthrobacter crystallopoietes (WP-005270754), Bosea psychrotolerans (WP-181011807), Glaciecola amylolytica (WP-164472126), Hyphomonas sp. (HA037884), Janthinobacterium psychrotolerans (WP-065307954), Massilia psychrophile (WP-099914383), Paraglaciecola psychrophile (WP-007642709), Polaromonas sp. SP1 (WP-164483751), Polaromonas sp. AER18D-145 (WP-096697750), Polaromonas sp. CF318 (WP-007872516), Polaromonas vacuolate (WP-168920719), Polaromonas naphthalenivorans (WP-157040436), Polaromonas sp. JS666 (WP-011482994), Polaromonas glacialis (WP-084181426), Polaromonas sp. EUR3 1.2.1 (WP-197028649), Polaromonas sp. CG_9.2 (WP-196864241), Polaromonas sp. CG_9.11 (WP-196869863), Polaromonas eurypsychrophila (WP-188708524), Polaromonas sp. (MBC7445758), Polaromonas jejuensis (WP-068832216), Polaromonas sp. AET17H-212 (WP-096671180), Polaromonas sp. YR568 (WP-092127764), Polaromonas sp. C04 (WP-077562980), Pseudorhodobacter psychrotolerans (WP-08235149), Psychrobacillus lasiicapitis (WP-142537823), Psychrobacillus sp. OK032 (WP-093265425), Psychrobacillus sp. OK028 (WP-093060398), Psychrobacillus sp. FJAT-21963 (WP-056833301), Psychrobacter jeotgali (WP-201583776), Psychrobacter sp. H8-1 (WP-201574875), Psychrobacter sp. Cmf 22.2 (WP-075103245), Psychrobacter sp. ENNN9_III (WP-058368887), Psychrobacter sp. P2G3 (WP-068327306), Psychrobacter sp. P11G5 (WP-068035467), Psychrosphaera haliotis (WP-155693683), Shewanella psychrophile (WP-077755816), Simplicispira psychrophile (WP-051603004), Sphingobium psychrophilum (WP-169570392), Sphingomonas psychrolutea (WP-188445826), Clostridium homopropionicum (WP-074782965), Clostridium sp. DL-VIII (WP-009169886), Clostridium clostridioforme CAG:132 (CDB63357), Zunongwangia atlantica 221114-10F7 (ORL47196).

Extremophilic enzymes produced by halophiles can be utilized in some embodiments. For instance, halophilic enzymes that exhibit activity at a salinity of about 1 M or greater, about 2 M or greater in some embodiments, can be utilized. Exemplary halophiles (and halophilic enzymes produced thereby) encompassed herein can include, without limitation, Alteromonas halophila (WP-189403400), Arthrobacter crystallopoietes (WP-005270754), Arthrobacter sp. NEB 688 (WP-173027059), Azospirillum halopraeferens (WP-029007775), Empedobacter haloabium (TXE30443), Desulfovibrio sulfodismutans (NDY59052), Halobacillus hunanensis (WP-139377117), Halobacillus ihumii (WP-16352794), Halobacteriovorax marinus (WP-157868258), Haloechinothrix halophila (WP-051400222), Halomarina oriensis (WP-158204529), Halomonas cerina (WP-183325502), Halomonas korlensis (WP-089794761), Halomonas sp. PR-M31 (WP-048308188), Halomonas aquamarine (WP-089674669), Halomonas zhanjiangensis (WP-040460201), Halomonas aestuarii (WP-071946866), Halomonas endophytica (WP-102654199), Halomonas heilongjiangensis (WP-102629242), Halomonas campaniensis (WP-088701082), Halomonas alkaliphile (WP-038486873), Halomonas sp. ALS9 (WP-064233856), Halomonas sp. GFAJ-1 (WP-009098816), Halomonas sp. KHS3 (WP-041159480), Halomonas alkaliphile (WP-162218603), Halomonas sp. ZH2S (WP-160419650), Halomonas alkaliantarctica (WP-133732469), Halomonas zincidurans (WP-031384106), Halomonas chromatireducens (WP-083517585), Halomonas sp. KO116 (WP-035563078), Halmonas sp. A40-4 (WP-199285424), Halomonas ventosae (WP-035579360), Halomonas sp. HAL1) WP-008958555), Halomonas sp. MES3-P3E (WP-101146070), Halomonas sp. 1513 (WP-083700770), Halomonas sp. GT (WP-083007892), Halomonas sp. PA5 (QJQ97022), Halomonas songnenensis (WP-106373458), Halomonas subglaciescola (WP-079553041), Halomonas sp. HL-92 (WP-074398447), Halomonas xinjiangensis (WP-197053288), Halomonas saliphila (WP-104202516), Halomonas sp. HL-48 (WP-027336292), Halomonas qijiaojingensis (WP-189471950), Halomonas urumqiensis (WP-102588859), Halomonaslutea (WP-019020614), Halomonas lutescens (WP-188638020), Halomonas salicampi (WP-179930793), Halomonas sp. FME66 (WP-193092800), Halomonas sp. 156 (CAD5269671), Halomonas sp. L5 (WP-149329933), Halomonas nanhaiensis (WP-127060197), Halomonas titanicae (WP-144810212), Halomonas sp. SH5A2 (WP-186255949), Halomonas sp. TD01 (WP-009722522), Halomonas sp. PC (WP-127040515), Halomonas sp. RC (WP-126951333), Halomonas sp. DQ26W (WP-114573011), Halomonas sp. TQ8S (WP-114486842), Halomonas sp. PYC7W (WP-114478819), Halomonas sp. LBP4 (WP-181421925), Halomonas sp. QX-1 (WP-176303735), Halomonas sp. QX-2 (WP-180092182), Halomonas glaciei (WP-179915254), Halomonas zhaodongensis (WP-179927495), Halomonas xianhensis (WP-092845804), Halomonas gudaonensis (WP-089686750), Halomonas humidisoli (WP-095603093), Halomonas boliviensis (WP-083825729), Halomonas sp. QHL1 (WP-083571058), Halomonas ilicicola (WP-072822829), Halomonas saccharevitans (WP-089847692), Halomonas muralis (WP-089729617), Halomonas arcis (WP-089706930), Halomonas boliviensis (WP-040480056), Halomonas andesensis (WP-126944084), Halomonas sp. G5-11 (WP-168017113), Halomonas sp. THAF5a (QFU03326), Halomonas taeanensis (SDG32001), Halorussus sp. RC-68 (WP-128475846), Halorussus ruber (WP-135825713), Halorussus sp. ZS-3 (WP-158056449), Halorussus sp. HD8-83 (WP-135830119), Halorussus salinus (WP-135854680), Halorussus amylolyticus (WP-132060623), Halorussus sp. MSC15.2 (WP-163523881), Haloterrigena limicola (WP-008010666), Haloterrigena hispanica (WP-149782231), Haloterrigena sp. H1 (WP-138782397), Isoptericola halotolerans (WP-171781920), Marinobacter sp. X15-166B (WP-198929205), Marinobacter sp. LPB0319 (WP-2066439888), Marinobacter salaries (WP-126811858), Marinobacter sp. PJ-16 (WP-137435339), Marinobacter nanhaiticus (WP-004579452), Marinobacter bohaiensis (WP-111497193), Marinobacter sp. ANT_B65 (WP-202971753), Marinobacter sediminum (WP-203299860), Marinobacter fonticola (WP-148861082), Marinobacter sp. JB02H27 (WP-150989051), Marinobacter maritimus (WP-144775354), Marinobacter nitratireducens (WP-036130189), Marinobacter aromaticivorans (WP-100686899), Marinobacter sp. MCTG268 (WP-081899301), Marinobacter profundi (WP-099614009), Marinobacter sp. R17 (WP-123633665), Marinobacter sp. F3R11 (WP-113816648), Marinobacter lipolyticus(WP-012136507), Marinobacter sp. LV10MA510-1 (WP-098421792), Marinobacter sp. LV10R520-4 (WP-143751449), Marinobacter antarcticus (WP-072795398), Marinobacter zhejiangensis (WP-092022278), Marinobacter sp. LZ-8 (WP-138439039), Marinobacter sp. LZ-6 (WP-138437074), Marinobacter sp. DS40M8 (WP-169052525), Marinobacter shengliensis (WP-106694886), Marinobacter algicola (WP-007152654), Marinobacter salicampi (WP-166253549), Marinobacter sp. JSM 1782161 (WP-165857264), Methyloligella halotolerans (WP-069095898), Micromonospora halophytica (WP-091291516), Natronococcus sp. LS1_42 (WP-148858780), Nocardiopsis halotolerans (WP-017570132), Paracoccus halophilus (WP-036743786), Roseivivax halodurans (WP-037257008), Saccharomonospora halophila (WP-157601674), Shewanella vesiculosa (NC072699), Shewanella psychrophila (WP-077755816), Shewanella frigidimarina (WP-123883413), Shewanella khirikhana (WP-126168307), Shewanella halifaxensis (WP-108946642), Shewanella waksmanii(WP-028774143), Shewanella saliphila (WP-188922486), Shewanella ulleungensis (WP-188954542), Shewanella litoralis (WP-160052797).

Extremophilic enzymes produced by acidophiles can be utilized in some embodiments. For instance, acidophilic enzymes that exhibit activity at a pH of from about 1 to about 5.5 can be utilized. Exemplary acidophiles (and acidophilic enzymes produced thereby) encompassed herein can include, without limitation, Acidibrevibacterium fodinaquatile (WP-162800754), Acidicaldus sp (HGC43174), Acidiphilium cryptum (WP-050751056), Acidisphaera rubrifaciens (WP-084623200), Acidisphaera sp. S103 (WP-158926549), Acidobacteria bacterium (MB14850940), Acidobacteriales bacterium (MBA3914351), Acidimicrobiaceae bacterium (TPW09344), Acidothermus cellulolyticus (WP-011719018), Acidovorax sp. (RZJ59385), Acidovorax sp. Leaf160 (WP-156382378), Acidovorax citrulli (WP-116212334), Acidovorax sp. ST3 (WP-110960035), Acidovorax sp. SD340 (WP-055393692), Acidovorax sp. JHL-9 (WP-026434583), Acidovorax sp. JHL-3 (WP-024815995), Acidovorax sp. 59 (WP-099731663), Acidovorax sp. T1 (WP-087747071), Acidovorax radices (WP-145694120), Acidovorax citrulli (MVT28077), Acidovorax konjaci (WP-184273732), Acidovorax sp. YL-MeA13-2016 (WP-179683865), Acidovorax sp. JMULE5 (WP-176888736), Acidovorax carolinensis (WP-086926820), Acidovorax sp. Root219 (WP-057264729), Acidovorax sp. Root217 (WP-057200451), Acidovorax sp. Root70 (WP-056639581), Acidovorax sp. Root267 (WP-057271450), Acidovorax sp. Root275 (WP-057228519), Acidovorax sp. Root568 (WP-056742554), Acidovorax sp. Root402 (WP-056056880), Acidovorax sp. Leaf78 (WP-056167938), Acidovorax sp. CF316 (WP-007848954), Acidovorax sp. NO-1 (WP-008904688), Acidovorax sp. KKS102 (WP-015015374), Acidovorax sp. BoFeN1 (WP-114656624), Acidovorax sp. MR-S7 (WP-020227330), Acidovorax sp. GW101-3H11 (WP-063462297), Acidovorax sp. 100 (WP-121942233), Acidovorax sp. 94 (WP-121421729), Acidovorax sp. 93 (WP-121508058), Acidovorax sp. IB03 (WP-198847087), Acidovorax facilis (WP-182119389), Acidovorax cattleya (WP-196290774), Acidovorax soli (WP-184855240), Acidovorax sp. TP4 (BAA35137), Acidovorax sp. HMWF018 (WP-199227795), Acidovorax sp. 107 (WP-108624875), Acidovorax sp. 69 (WP-100412617), Acidovorax sp. RAC01 (WP-069104250), Acidovorax avenae (WP-107129247), Acidovorax sp. ACV01(WP-192426852), Acidovorax sp. ACV02 (WP-192419383), Acidovorax sp. SRB_14 (WP-173025722), Acidovorax sp. 99 (WP-116748450), Acidovorax delafieldii (WP-060985808), Acidovorax sp. 16-35-5 (WP-175506463), Acidovorax valerianellae (WP-092740663), Acidovorax temperans (WP-142084895), Acidovorax oryzae (WP-026433360), Acidovorax sp. SRB_24 (WP-169168665), Acidovorax cavernicola (WP-119555154), Acidovorax temperans (WP-044398345), Acidisoma sp. S159 (WP-159014448), Acidisoma sp. L85 (WP-158802619), Acidisphaera sp. L21 (WP-158747166), Acidiphilium cryptum JF-5 (ABQ28771), Actinospica acidiphila (WP-193455356), Alicyclobacillus pomorum (WP-084453829), Amycolatopsis acidiphila (WP-144638401), Azospirillum baldaniorum (WP-014240680), Bacillus megaterium (WP-013057692), Catenulispora acidiphila (WP-015793547), Delftia sp. UME58 (WP-183018265), Delftia acidovorans (WP-202760212), Delftia lacustris (WP-016453321), Methylocapsa acidiphila (WP-026607232), Paraburkholderia acidophila (WP-084908171), Paraburkholderia acidisoli (WP-158957882), Paraburkholderia acidipaludis (WP-027796272), Priestia megaterium (WP-016764703), Rhizobium acidisoli (WP-054183259), Rhodoblastus acidophilus (WP-088519736), Stenotrophomonas acidaminiphila (WP-054666853), Streptomyces acidiscabies (WP-078480871), Streptomyces acidicola (WP-152864677).

Extremophilic enzymes produced by alkaliphiles can be utilized in some embodiments. For instance, alkaliphilic enzymes that exhibit activity at a pH of from about 7.5 to about 11.5) can be utilized. Exemplary alkaliphiles (and alkaliphilic enzymes produced thereby) encompassed herein can include, without limitation, Alkalilacustris brevis (WP-114966465), Alkalihalobacillus macyae (WP-152670966), Alkalihalobacillus pseudofirmus (WP-012960136), Alkalihalobacillus shacheensis (WP-082676287), Alkalihalobacillus xiaoxiensis (WP-204463621), Alkalilimnicola sp. S0819 (WP-152144452), Alkalimonas amylolytica (WP-091344878), Amycolatopsis alkalitolerans (WP-139096058), Cupriavidus alkaliphilus (WP-111516860), Ensifer alkalisoli (WP-151613639), Lacimicrobium alkaliphilum (WP-062478888), Lysobacter alkalisoli (QDH70273), Massilia alkalitolerans (WP-036214799), Methylobacter sp. B2 WP-174627553), Neorhizobium alkalisoli (WP-105385441), Nocardiopsis alkaliphile (WP-051045978), Ramlibacter alkalitolerans (WP-201687394), Spinactinospora alkalitolerans (WP-179641803).

Extremophilic enzymes produced by piezophiles can be utilized in some embodiments. For instance, piezophilic enzymes that exhibit activity at a pressure of about 110 kPa or greater, or about 50 MPa or greater in some embodiments, can be utilized. Exemplary piezophiles (and piezophilic enzymes produced thereby) encompassed herein can include, without limitation, Oceanobacillus piezotolerans (WP-121525044), Oceanobacillus profunda (WP-169713018), Colwellia marinimaniae (WP-082606415), Salinimonas sediminis (WP-108566897).

Radiation resistant extremophiles are also encompassed herein. For instance radiation resistant organisms such as Deinococcus radiotolerans which produces a radiation resistant extremozyme (WP_189068351) can be utilized. A radiation resistant organism and radiation resistant extremozyme encompassed herein can generally be active at a level of acute ionizing radiation (gamma rays, high energy UV rays, X-rays, etc.) of about 1000 Gy or greater, or about 2000 Gy or greater in some embodiments.

Extremozymes for use as disclosed need not necessarily be confined to those that naturally occur in an ‘extremely’ extreme environment. For example, it is not necessary to utilize an extremozyme produced by one of the most halophilic bacterium/archaea that exists (e.g., Salinibacter ruber, which exists at a 5.0 M saline microenvironment). Rather, disclosed systems and methods can include an extremozyme produced by an organism that exists in an environment at which pathogenic mesophiles cannot live or grow (e.g., about 1.0 M NaCl). Thus extremozymes as encompassed herein include those produced by organisms that can survive at moderately extreme sources.

In some embodiments, PHBDase/bacterium/archaea for use as disclosed can include polyextremophiles and/or polyextremozymes that exist at a combination of two or more extreme environmental conditions. For example a halophilic alkalithermophile, which ideally exist at high saline, temperature, and alkaline conditions, or a psychrotrophic halophile, which ideally exist at both low temperature and high saline conditions. Most of the piezophilic (pressure-loving) extremophiles are found at the bottom of the ocean and are therefore also halophilic (salt-loving) and psychrophilic (cold-loving), all of which are conditions that can be simultaneously generated and maintained within a reaction chamber to provide mesophilic pathogen decontamination. In such an embodiment, mesophilic contamination can be addressed through multiple mechanisms in conjunction with a depolymerization reaction catalyzed by a single polyextremozyme.

In some embodiments, utilization of a plurality of extreme conditions for decontamination in conjunction with enzymatic biodegradation by use of one or more polyextremozymes can provide for an increase in pathogen death rate as compared to utilization of a single extreme condition. For instance, the death rate of a pathogen during a depolymerization/decontamination process under multiple extreme conditions can be greater than the death rate of the pathogen under any one of the extreme conditions alone, and in some embodiments, the pathogen death rate can be greater than the sum of the death rates under the individual extreme conditions. Moreover, in some embodiments, utilization of multiple extreme conditions during a procedure can increase the rate of depolymerization, e.g., the production of monomer or oligomer hydroxyalkanoate, as compared to the depolymerization rate under a single extreme condition.

Improved decontamination effectiveness under multiple extreme conditions can be beneficial in embodiments in which relatively moderate extreme conditions are desired. For instance, through utilization of one or more polyextremozymes (optionally including one or more single condition extremozymes) in a depolymerization/decontamination operation, a combination of relatively moderate extreme conditions (e.g., salinity of about 1.0 M NaCl combined with a relatively moderate increased temperature of about 50° C. to about 80° C.) can provide for rapid decontamination and depolymerization at a lower cost than a single extreme condition system at higher salinity or temperature.

A number of the extremophiles and extremozymes previously mentioned are polyextremophiles. Table 1, below, provides a non-limiting listing of polyextremophile genera that express a PHBDase as may be utilized as described herein. The Actinobacteria is a phylum of bacterial that thrive at both high temperature and alkaline environments, and taxonomic family members of this phylum (e.g., Streptosporangiceae, Thermomonosporaceae, Nocardiopsaceae, Bogoriellaceae, Streptomycetaceae, and Pseudoonocardiaceae) can be utilized in some embodiments. Of course, acidic conditions can likewise be combined with temperature or other extreme conditions. Examples of polyextromophilic bacteria that are thermoacidophiles include, without limitation, Acidothermaceae, Acidimicrobiaceae, Thermoleophilaceae, and Rubrobacteraceae.

TABLE 1

| Genus | Nature of Extreme Conditions |
|---|---|
| Acidothermus | Thermophile, pH |
| Alicyclobacillus | Thermophile, pH |
| Amycolatopsis | Thermophile, pH |
| Arthrobacter | Psychrohile, pH |
| Colwellia | Psychrophile, Piezophile |
| Georgenia | Thermophile, pH |
| Halobacillus | Halophile, pH |
| Halobacteriaceae* | Halophile, Peizophile, Psychrophile |
| Halomonas | Psychrophile, Halophile, pH, Piezophile |
| Marinobacter | Psychrophile, Halophile |
| Natronococcus | Halophile, pH, Thermophile |
| Thermobifida | Thermophile, pH, Halophile |
| Zunongwangia | Psychrophile, Halophile |

*Taxonomic family

Exemplary polyextremophiles (and polyextremophilic enzymes produced thereby) encompassed herein can include, without limitation (some of which are also included in those referred to previously), Acidothermus cellulolyticus (WP_011719018), Arthrobacter crystallopoietes (WP_005270754), Arthrobacter sp. NEB 688 (WP_173027059), Amycolatopsis decaplanina (WP_007028471), Amycolatopsis azurea (WP_039919726), Amycolatopsis orientalis (WP_044853678), Amycolatopsis regifaucium (WP_061985795), Amycolatopsis alba (WP_020632115), Amycolatopsis sp. CB00013 (WP_073845662), Amycolatopsis sp. WAC 04182 (WP_125683401), Amycolatopsis sp. WAC 04197 (WP_125733174), Amycolatopsis sp. WAC 01416 (WP_125797595), Amycolatopsis lurida (WP_034314791), Amycolatopsis australiensis (WP_072479564), Amycolatopsis sp. WAC 01375 (WP_125786221), Amycolatopsis sp. YIM 10 (WP_194239921), Amycolatopsis australiensis (WP_072480012), Amycolatopsis sp. WAC 01376 (WP_125797552), Amycolatopsis sp. WAC 01376 (WP_125791151), Amycolatopsis sp. BJA-103 (WP_168214428), Amycolatopsis sp. WAC 04169 (WP_125694889), Amycolatopsis sp. YIM 10 (WP_153034611), Amycolatopsis xylanica (WP_091289432), Amycolatopsis thailandensis (WP_093938547), Amycolatopsis tolypomycina (WP_091314877), Amycolatopsis (WP_094002797), Amycolatopsis mediterranei (WP_013227677), Amycolatopsis tolypomycina (WP_091316988), Amycolatopsis mediterranei (WP_013225900), Amycolatopsis sp. MJM2582 (WP_037335097), Amycolatopsis pretoriensis (WP_086680613), Amycolatopsis mediterranei (WP_014467631), Amycolatopsis mediterranei (WP_013227743), Amycolatopsis lexingtonensis (WP_086861387), Amycolatopsis balhimycina (WP_026468360), Amycolatopsis tolypomycina (WP_091309318), Amycolatopsis mediterranei (WP_013225589), Amycolatopsis lexingtonensis (WP_086864508), Amycolatopsis balhimycina (WP_020640708), Amycolatopsis balhimycina (WP_020639925), Amycolatopsis japonica (WP_038521005), Amycolatopsis vancoresmycina (WP_051767789), Amycolatopsis vancoresmycina (WP_162146255), Amycolatopsis vancoresmycina (WP_003055279), Amycolatopsis vancoresmycina (WP_003059137), Amycolatopsis arida (WP_177216885), Amycolatopsis orientalis (WP_037305638), Amycolatopsis mediterranei U32 (ADJ49174), Amycolatopsis balhimycina (WP_020640186), Amycolatopsis balhimycina (WP_020646797), Amycolatopsis regifaucium (WP_158070237), Amycolatopsis umgeniensis (WP_184896802), Amycolatopsis mediterranei (WP_176742238), Amycolatopsis orientalis (WP_037318494), Amycolatopsis taiwanensis (WP_027941815), Amycolatopsis thermoflava (WP_037323546), Amycolatopsis nigrescens (WP_157357235), Amycolatopsis benzoatilytica (WP_020658806), Amycolatopsis thermoflava (WP_123687648), Amycolatopsis sp. MtRt-6 (WP_206788940), Amycolatopsis nigrescens (WP_020673950), Amycolatopsis sp. MtRt-6 (WP_206796628), Amycolatopsis sp. MtRt-6 (WP_206785025), Amycolatopsis sp. 195334CR (WP_206808196), Amycolatopsis sp. SID8362 (WP_166641473), Amycolatopsis vastitatis (WP_167441766), Amycolatopsis sp. MtRt-6 (WP_206794433), Amycolatopsis sp. 195334CR (WP_206804625), Amycolatopsis sp. SID8362 (WP_160695402), Amycolatopsis sp. 195334CR (WP_206805671), Amycolatopsis mediterranei S699 (AEK42609), Amycolatopsis sp. SID8362 (WP_160697844), Amycolatopsis ruanii (WP_116109633), Amycolatopsis vastitatis (WP_093953441), Amycolatopsis antarctica (WP_094864937), Amycolatopsis sp. SID8362 (WP_160697847), Amycolatopsis vastitatis (WP_093953193), Amycolatopsis rifamycinica (WP_043779284), Amycolatopsis rifamycinica (WP_043787922), Amycolatopsis orientalis (WP_044854926), Amycolatopsis albispora (WP_113697064), Amycolatopsis vastitatis (WP_093953762), Amycolatopsis keratiniphila (WP_043848437), Amycolatopsis rifamycinica (WP_043776526), Amycolatopsis sp. ATCC 39116 (WP_039791697), Amycolatopsis sp. CA-126428 (WP_199191631), Amycolatopsis sp. CA-128772 (WP_199199004), Amycolatopsis rifamycinica (WP_043775110), Amycolatopsis sp. CA-128772 (WP_103347542), Amycolatopsis sp. CA-126428 (WP_103341161), Amycolatopsis sp. CA-126428 (WP_103338297), Amycolatopsis sp. CA-128772 (WP_103347494), Amycolatopsis sp. CA-128772 (WP_103351389), Amycolatopsis sp. CA-126428 (WP_10334050), Amycolatopsis sp. CA-126428 (WP_103337215), Amycolatopsis sp. BJA-103 (WP_101611121), Amycolatopsis rifamycinica (WP_043775220), Amycolatopsis bullii (WP_191309718), Amycolatopsis alkalitolerans (WP_139096058), Amycolatopsis sp. CA-126428 (WP_103340450), Amycolatopsis sp. A23 (WP_155542679), Amycolatopsis sp. A23 (WP_155546301), Amycolatopsis bullii (WP_191313482), Amycolatopsis oliviviridis (WP_191256639), Amycolatopsis bullii (WP_191317041), Amycolatopsis sp. A23 WP_155546374), Amycolatopsis bullii (WP_191309628), Amycolatopsis sp. H6(2020) (MBE8525409), Amycolatopsis sp. H6(2020) (MBE8516875), Amycolatopsis acidiphila (WP_144638401), Amycolatopsis deserti (WP_191242759), Amycolatopsis sp. H6(2020) (MBE8523464), Amycolatopsis roodepoortensis (WP_192744003), Amycolatopsis lexingtonensis (WP_086861614), Amycolatopsis sp. H6(2020) (MBE8523449), Amycolatopsis lexingtonensis (WP_086861672), Amycolatopsis sp. H6(2020) (MBE8519699), Amycolatopsis eburnean (WP_125314097), Amycolatopsis sp. PIP199 (WP_181777181), Amycolatopsis eburnean (WP_125313793), Amycolatopsis sp. YIM 10 (WP_153034239), Amycolatopsis rhizosphaerae (WP_144585784), Amycolatopsis eburnea (WP_191984376), Amycolatopsis australiensis (WP_072479963), Amycolatopsis eburnea (WP_125313723), Amycolatopsis sp. Hca4 (WP_176178332), Amycolatopsis pretoriensis (WP_086674376), Amycolatopsis sp. YIM 10 (WP_153033440), Amycolatopsis sp. Hca4 (WP_176171164), Amycolatopsis thermalba (WP_115944128), Amycolatopsis tolypomycina (WP_091313624), Amycolatopsis sacchari (WP_09150482), Amycolatopsis kentuckyensis (WP_086849953), Amycolatopsis pretoriensis (WP_086676731), Amycolatopsis kentuckyensis (WP_086838850), Amycolatopsis vancoresmycina (WP_033262149), Amycolatopsis sacchari (WP_091509483), Amycolatopsis eburnea (RSD12104), Amycolatopsis vancoresmycina (WP_033262457), Amycolatopsis tolypomycina (WP_091314771), Amycolatopsis kentuckyensis (WP_086842561), Amycolatopsis tolypomycina (SED02538), Amycolatopsis kentuckyensis (WP_086850817), Amycolatopsis keratiniphila (SDU59319), Amycolatopsis sp. SID8362 (NBH10816), Amycolatopsis sacchari (SF91313), Amycolatopsis keratiniphila (AGM10176), Amycolatopsis vancoresmycina DSM 44592 (EOD69417), Amycolatopsis vancoresmycina DSM 44592 (EOD63279), Colwellia psychrerythraea (WP_033095470), Colwellia psychrerythraea (WP_033082346), Colwellia chukchiensis (WP_085285385), unclassified Colwellia (WP_182245161), unclassified Colwellia (WP_108456828), Colwellia (WP_082606415), unclassified Colwellia (WP_182136131), unclassified Colwellia (WP_182222214), Colwellia psychrerythraea (WP_138140233), unclassified Colwellia (WP_182213899), unclassified Colwellia (WP_182191078), Colwellia psychrerythraea (WP_033082290), Colwellia sp. Arc7-635 (WP_126668020), Colwellia aestuarii (WP_143323591), Colwellia sp. BRX8-4 (WP_182258889), Colwellia sp. (MBL4900302), Colwellia sp. (MBL0710453), Colwellia sp. PAMC 21821 (WP_081180401), Colwellia sp. (MBL4764635), Colwellia sp. 12G3 (WP_101233926), Colwellia polaris (WP_085306422), Colwellia sp. Bg11-28 (WP_157825823), Colwellia sp. BRX10-3 (WP_182133028), Colwellia sp. MB02u-6 (WP_182233718), Colwellia sp. BRX8-2 (WP_182231462), Colwellia sp. MB3u-4 (WP_182185277), Colwellia sp. BRX9-1 (WP_182230151), Colwellia sp. BRX8-7 (WP_182242732), Colwellia sp. (NQZ90610), Colwellia sp. MB02u-10 (WP_182238471), Colwellia sp. (NQZ28611), Colwellia sp. (QY47923), Colwellia sp. Bg11-12 (WP_182229555), Colwellia sp. (NQY89088), Colwellia beringensis (WP_081152231), Colwellia sp. (NQZ82584), Colwellia demingiae (WP_146789187), Candidatus Colwellia aromaticivorans (WP_114327742), Colwellia sp. MB02u-9 (WP_182197537), Colwellia mytili (WP_085299583), Colwellia sp. (NQY47915), Colwellia sp. (NQZ28619), Haladaptatus paucihalophilus (WP_007977720), Haladaptatus litoreus (WP_076429835), Haladaptatus paucihalophilus (WP_007977722), Haladaptatus sp. R4 (WP_066143160), Haladaptatus cibarius (WP_049970104), Haladaptatus sp. (W1 WP_069450211), Haladaptatus cibarius (WP_049971911), Haladaptatus paucihalophilus DX253 (SHK49397), Halobacillus ihumii (WP_163527944), Halobacillus hunanensis (WP_139377117), Halomarina oriensis (WP_124957125), Halomarina oriensis (WP_158204529), Halomonas (ventosae) (WP_035579360), Halomonas sp. 156 (CAD5269671), unclassified Halomonas (WP_008956714), Halomonas (WP_035577590), Halomonas chromatireducens (WP_083517585), Halomonas meridiana (WP_083602247), unclassified Halomonas (sp. HL-92) (WP_074398447), Halomonas sp. GFAJ-1 (WP_009101808), Halomonas chromatireducens (WP_066448186), Halomonas sp. KO116 (WP_035563078), Halomonas sp. KO116 (WP_035565981), Halomonas arcis (WP_089708323), Halomonas sp. TD01 (WP_009724586), Halomonas arcis (WP_089706930), Halomonas korlensis (WP_089792833), Halomonas alkaliantarctica (WP_133732469), Halomonas ilicicola (WP_072822829), Halomonas boliviensis (WP_007114283), Halomonas sp. HL-48 (WP_027336292), Halomonas alkaliphila (WP_038486873), unclassified Halomonas (WP_074394764), Halomonas sp. HAL1 (WP_008958555), Halomonas subglaciescola (WP_079553041), Halomonas korlensis (WP_089797758), Halomonas cerina (WP_183325502), unclassified Halomonas (sp. RC) (WP_126951333), Halomonas sp. TD01 (WP_009722522), Halomonas titanicae (WP_089691351), Halomonas aquamarina (WP_089674669), Halomonas gudaonensis (WP_089686750), Halomonas alkaliantarctica (WP_133731111), Halomonas saccharevitans (WP_089847692), Halomonas xianhensis (WP_092845804), Halomonas songnenensis (WP_106373458), Halomonas zincidurans (WP_031384106), Halomonas lutea (WP_019020614), Halomonas boliviensis (WP_083825729), Halomonas sp. GFAJ-1 (WP_009098816), Halomonas muralis (WP_089729617), Halomonas boliviensis (WP_040480056), Halomonas sp. (HAA45741), Halomonas zhanjiangensis (WP_040460201), Halomonas campaniensis (WP_088701082), Halomonas alkaliphile (WP_162218603), Halomonas sp. ZH2S (WP_160419650), Halomonas endophytica (WP_102654199), Halomonas sp. ALS9 (WP_064233856), Halomonas sp. KHS3 (WP_041159480), Halomonas salicampi (WP_179930793), Halomonas salicampi (WP_179928774), Halomonas heilongjiangensis (WP_102629242), Halomonas campaniensis (WP_088701419), Halomonas sp. MES3-P3E (WP_101146070), Halomonas alkaliantarctica (WP_030070137), Halomonas xinjiangensis (WP_197053288), Halomonas alkaliantarctica (WP_030072571), Halomonas sp. GT (WP_083002052), Halomonas sp. A40-4 (WP_199285424), Halomonas sp. GT(WP_083007892), Halomonas sp. 1513 (WP_076746720), Halomonas sp. HL-48 (WP_027335517), Halomonas sp. 1513 (WP_083700770), Halomonas sp. (MBL1266350), Halomonas urumqiensis (WP_102588859), Halomonas lutescens (WP_188638020), Halomonas lutescens (WP_188638515), Halomonas sp. FME66 (WP_193092800), Halomonas saliphila (WP_104202516), Halomonas sp. (MBE0488383), Halomonas qijiaojingensis (WP_189471950), Halomonas sp. 3(2) (WP_151442249), Halomonas sp. FME20 (WP_192536925), Halomonas sp. SH5A2 (WP_186255949), Halomonas sp. TQ8S (WP_114486842), Halomonas titanicae (WP_144812651), Halomonas sp. PYC7W (WP_114478819), Halomonas sp. PYC7W (WP_114478692), Halomonas sp. LBP4 (WP_181421925), Halomonas sp. TQ8S (WP_114487405), Halomonas glaciei (WP_179915254), Halomonas sp. QX-2 9 (WP_180092182), Halomonas sp. SH5A2 (WP_186253301), Halomonas zhaodongensis (WP_179927495), Halomonas titanicae (WP_144810212), Halomonas nanhaiensis (WP_127060197), Halomonas pantelleriensis (WP_089659512), Halomonas zhaodongensis (WP_179926908), Halomonas humidisoli (WP_095603093), Halomonas sp. QHL1 (WP_083571058), Halomonas sp. PC (WP_127040515), Halomonas sp. DQ26W (WP_114573011), Halomonas shengliensis (WP_089679049), Halomonas sp. QX-1 (WP_176303735), Halomonas sp. QHL1 (WP_071693265), Halomonas korlensis (WP_089794761), Halomonas aestuarii (WP_071946866), Halomonas sp. PR-M31 (WP_048308188), Halomonas sp. PA5 (QJQ97022), Halomonas andesensis (WP_126944084), Halomonas sp. PA5 (QJQ94877), Halomonas sp. L5 (WP_149329933), Halomonas korlensis (SFU56513), Halomonas sp. G5-11 (WP_168017113), Halomonas subterranean (WP_092824778), Halomonas sp. (HDZ47214), Halomonas sp. THAF5a (QFU03326), Halomonas sp (HDZ46744), Halomonas chromatireducens (AMD02558), Halomonas andesensis (WP_126948398), Halomonas korlensis (SFU93166), Halomonas taeanensis (SDG32001), Halorussus salinus (WP_135854385), Halorussus sp. MSC15.2 (WP_163523881), Halorussus salinus (WP_135854680), Halorussus amylolyticus (WP_132060623), Halorussus sp. ZS-3 (WP_158056449), Halorussus amylolyticus (WP_132060625), Halorussus sp. ZS-3 (WP_158056448), Halorussus sp. RC-68 (WP_128475846), Halorussus ruber (WP_135825713), Halorussus ruber (WP_135825712), Halorussus sp. HD8-83 (WP_135830119), Marilnobacter sp. LV10R520-4 (WP_143751449), Marinobacter zhejiangensis (WP_092022278), unclassified Marinobacter (WP_150989051), Marinobacter nitratireducens (WP_036130189), Marinobacter salarius (WP_091640839), unclassified Marinobacter (WP_098419392), Marinobacter algicola (WP_007152654), Marinobacter antarcticus (WP_072795398), unclassified Marinobacter (WP_152438805), Marinobacter (WP_075197007), Marinobacter profundi (WP_099614009), Marinobacter sp. LPB0319 (WP_206643988), Marinobacter sp. DS40M8 (WP_169052525), Marinobacter sp. X15-166B (WP_198929205), unclassified Marinobacter (WP_081899301), Marinobacter sp. PJ-16 WP_137435339), Marinobacter bohaiensis (WP_111497193), Marinobacter sediminum (WP_203299860), Marinobacter lipolyticus (WP_012136507), Marinobacter sp. ANT_B65 (WP_202971753), Marinobacter nanhaiticus (WP_004579452), Marinobacter salarius (WP_126811858), Marinobacter maritimus (WP_144775354), Marinobacter sp. F3R11 (WP_113816648), Marinobacter sp. LZ-8 (WP_138439039), Marinobacter sp. LZ-6 (WP_138437074), Marinobacter shengliensis (WP_106694886), Marinobacter fonticola (WP_148861082), Marinobacter sp. JSM 1782161 (WP_165857264), Marinobacter sp. R17 (WP_123633665), Marinobacter salicampi (WP_166253549), Marinobacter sp. LV10MA510-1 (WP_098421792), Thermobifida fusca (WP_016187994), Zunongwangia atlantica 221114-10F7 (ORL471960).

Of course, any combination of extremophiles and extremophilic enzymes can be utilized in disclosed methods and systems, and any combination of environmental conditions corresponding to active conditions for the extremozymes can likewise be utilized to provide a multi-dimensional approach to simultaneous decontamination of a post-consumer product and degradation of one or more biopolymers contained in the post-consumer product.

The selection of the extreme PHBDase/bacterium/archaea from a particular environment can be selected to match the needs of a depolymerization process. For instance if there is a need/desire to run a process at an elevated temperature then the PHBDase/bacterium/archaea can be selected from among the thermophiles. Similarly if there is a need or desire to run the reaction in the presence of high salt, then the PHBDase/bacterium/archaea for use can be selected from among the halophiles. Similarly, if less extreme conditions are required for the decontamination and depolymerization process, for instance due to the known contaminants, then extremophilic PHBDase/bacterium/archaea can be selected that exhibit high activity in those less extreme conditions. In such an embodiment, the decontamination process may not be required to be lethal to the pathogens, but may provide a less extreme approach to removing pathogenic characteristics from the contaminants.

As demonstrated in the examples section, below, when utilizing multiple extreme conditions, the bacterial death rate can generally be driven by the most extreme condition, and this can be utilized in selecting the extremozyme(s) of a process in order to obtain desired efficiency and balance between decontamination rate, depolymerization rate, and costs. Of course, while high pathogenic death rate can be a desirable factor on which to base process parameters, this may not be the most desirable factor in all conditions. For instance, when considering an industrial depolymerization process, the survivability half-life of the pathogen(s) may be utilized in selection of the PHAase rather than the overall pathogen death rate, generally in combination with the depolymerization rate of the enzyme, so as to most efficiently maximize all of depolymerization, decontamination, and costs of a process.

Disclosed methods and systems can utilize one or more natural extremozymes as expressed by an extremophilic microorganism and/or one or more extremophile(s) or genetically modified organisms that expresses the extremozyme(s) as well as genetically modified extremozymes. While extremozymes as expressed from a natural extremophile can be utilized in some embodiments, in some embodiments, an extremozyme for use as disclosed can be produced by a transgenic organism, for instance as an extremozyme that is produced in an industrial process by use of a transgenic high production organism. In such a case, the extremozyme can be essentially identical to a naturally-produced extremozyme or can include one or more modifications as compared to a natural extremozyme. Accordingly, a transformed cell or a cell-free expression system that can express an extremozyme as described is also encompassed herein.

An extremozyme can be expressed by transformation of a suitable host organism, for example, by use of either prokaryotic or eukaryotic host cells. Examples of host cell types include, without limitation, bacterial cells (e.g., E. coli), yeast cells (e.g., pichia, S. cerevisiae), cultured insect cell lines (e.g., Drosophila), plant cell lines (e.g., maize, tobacco, rice, sugarcane, potato tuber), mammalian cells lines (e.g., Chinese Hamster Ovary (CHO)). In one embodiment, a recombinant host cell system can be selected that processes and post-translationally modifies nascent polypeptides in a manner desired to produce the final catalytic enzyme.

A nucleic acid sequence that encodes an enzyme may be placed in an expression vector for expression in the selected host. Such expression vectors can generally comprise a transcriptional initiation region linked to the nucleic acid sequence that encodes the enzyme. An expression vector can also include a plurality of restriction sites for insertion of the nucleic acid to be under the transcriptional regulation of various control elements. The expression vector additionally may contain selectable marker genes. Suitable control elements such as enhancers/promoters, splice junctions, polyadenylation signals, etc. may be placed in close proximity to the coding region to permit proper initiation of transcription and/or correct processing of the primary transcript, i.e., the coding region for the enzyme. Alternatively, the coding region utilized in an expression vector may contain endogenous enhancers/promoters, splice junctions, intervening sequences, polyadenylation signals, etc., or a combination of both endogenous and exogenous control elements.

An expression vector generally includes in the 5′-3′ direction of transcription, a promoter, a transcriptional and translational initiation region, a DNA sequence that encodes the enzyme, and a transcriptional and translational termination region functional in the host cell. In one embodiment, a T7-based vector can be used, which can include at least the following components: an origin of replication, a selectable antibiotic resistance gene (e.g. —amp^{r}, tetr, chlrr), a multiple cloning site, T7 initiator and terminator sequences, a ribosomal binding site, and a T7 promoter.

In general, any suitable promoter may be used that is capable of operative linkage to the heterologous DNA such that transcription of the DNA may be initiated from the promoter by an RNA polymerase that may specifically recognize, bind to, and transcribe the DNA in an open reading frame. Some useful promoters include, constitutive promoters, inducible promoters, regulated promoters, cell specific promoters, viral promoters, and synthetic promoters. Moreover, while promoters may include sequences to which an RNA polymerase binds, this is not a requirement. A promoter may be obtained from a variety of different sources. For example, a promoter may be derived entirely from a native gene of the host cell, be composed of different elements derived from different promoters found in nature, or be composed of nucleic acid sequences that are entirely synthetic. A promoter may be derived from many different types of organisms and tailored for use within a given cell. For example, a promoter may include regions to which other regulatory proteins may bind in addition to regions involved in the control of the protein translation, including coding sequences.

A translation initiation sequence can be derived from any source, e.g., any expressed E. coli gene. Generally, the gene is a highly expressed gene. A translation initiation sequence can be obtained via standard recombinant methods, synthetic techniques, purification techniques, or combinations thereof, which are all well known. Alternatively, translational start sequences can be obtained from numerous commercial vendors. (Operon Technologies; Life Technologies Inc.).

The termination region may be native with the transcriptional initiation region, may be native with the coding region, or may be derived from another source. Transcription termination sequences recognized by the transformed cell are regulatory regions located 3′ to the translation stop codon, and thus together with the promoter flank the coding sequence. Examples include transcription termination sequences derived from genes with strong promoters, such as the trp gene in E. coli as well as other biosynthetic genes.

Vectors that may be used include, but are not limited to, those able to be replicated in prokaryotes and eukaryotes. For example, vectors may be used that are replicated in bacteria, yeast, insect cells, and mammalian cells. Examples of vectors include plasmids, phagemids, bacteriophages, viruses (e.g., baculovirus), cosmids, and F-factors. Specific vectors may be used for specific cells types. Additionally, shuttle vectors may be used for cloning and replication in more than one cell type. Such shuttle vectors are known in the art. The vector may, if desired, be a bi-functional expression vector that may function in multiple hosts.

An expression vector that encodes an extremozyme may be introduced into a host cell by any method known to one of skill in the art and the nucleic acid constructs may be carried extrachromosomally within a host cell or may be integrated into a host cell chromosome, as desired. A vector for use in a prokaryote host, such as a bacterial cell, includes a replication system allowing it to be maintained in the host for expression or for cloning and amplification. A vector may be present in the cell in either high or low copy number. Generally, about 5 to about 200, and usually about 10 to about 150 copies of a high copy number vector are present within a host cell. A host cell containing a high copy number vector will preferably contain at least about 10, and more preferably at least about 20 plasmid vectors. Generally, about 1 to 10, and usually about 1 to 4 copies of a low copy number vector will be present in a host cell.

In many embodiments, bacteria are used as host cells. Examples of bacteria include, but are not limited to, Gram-negative and Gram-positive organisms. In one embodiment an E. coli expression system suitable for T7 protein expression may be used. Examples of T7 expression strains can include, without limitation, BL21(DE3), BL21(DE3)pLysS, BLR(DE3)pLysS, Tuner(DE3)pLysS, Tuner(DE3), Lemo21(DE3), NiCO2(DE3), Oragami2(DE3), Origami B(DE3), Shuffle T7 Expres, HMS174(DE3), HMS174(DE3)pLysS, DH5aplhaE, Rosetta2(DE3), Rosetta2(DE3)pLysS, NovaBlue(DE3), Rosetta-gami B, Rosetta-gami B(DE3), Rosetta-gami B(DE3)pLysS, Rosetta Blue (DE3), Novagen(DE3), Novagen(DE3)pLysS.

An expression vector may be introduced into bacterial cells by commonly used transformation/infection procedures. A nucleic acid construct containing an expression cassette can be integrated into the genome of a bacterial host cell through use of an integrating vector. Integrating vectors usually contain at least one sequence that is homologous to the bacterial chromosome that allows the vector to integrate. Integrating vectors may also contain bacteriophage or transposon sequences. Extrachromosomal and integrating vectors may contain selectable markers to allow for the selection of bacterial strains that have been transformed.

Useful vectors for an E. coli expression system may contain constitutive or inducible promoters to direct expression of either fusion or non-fusion proteins. With fusion vectors, a number of amino acids are usually added to the expressed target gene sequence. Additionally, a proteolytic cleavage site may be introduced at a site between the target recombinant protein and the fusion sequence. Once the fusion protein has been purified, the cleavage site allows the target recombinant protein to be separated from the fusion sequence. Enzymes suitable for use in cleaving the proteolytic cleavage site include TEV, Factor Xa and thrombin. Fusion expression vectors which may be useful in the present can include those which express, for example and without limitation, Maltose Binding Protein (MBP), Thioredoxin (THX), Chitin Binding Domain (CBD), Hexahistadine tag (His-tag) (SEQ ID NO: 3), glutathione-S-transferase protein (GST), FLAG peptide, N-utilization substance (NusA), or Small ubiquitin modified (SUMO) fused to the target recombinant enzyme.

Methods for introducing exogenous DNA into a host cell are available in the art, and can include the transformation of bacteria treated with CaCl₂ or other agents, such as divalent cations and DMSO. DNA can also be introduced into host cells by electroporation, use of a bacteriophage, ballistic transformation, calcium phosphate co-precipitation, spheroplast fusion, electroporation, treatment of the host cells with lithium acetate or by electroporation. Transformation procedures usually vary with the bacterial species to be transformed.

Following transformation or transfection of a nucleic acid into a cell, the cell may be selected for the presence of the nucleic acid through use of a selectable marker. A selectable marker is generally encoded on the nucleic acid being introduced into the recipient cell. However, co-transfection of selectable marker can also be used during introduction of nucleic acid into a host cell. Selectable markers that can be expressed in the recipient host cell may include, but are not limited to, genes that render the recipient host cell resistant to drugs such as actinomycin Cl, actinomycin D, amphotericin, ampicillin, bleomycin, carbenicillin, chloramphenicol, geneticin, gentamycin, hygromycin B, kanamycin monosulfate, methotrexate, mitomycin C, neomycin B sulfate, novobiocin sodium salt, penicillin G sodium salt, puromycin dihydrochloride, rifampicin, streptomycin sulfate, tetracycline hydrochloride, and erythromycin. Selectable markers may also include biosynthetic genes, such as those in the histidine, tryptophan, and leucine biosynthetic pathways. Upon transfection or transformation of a host cell, the cell is placed into contact with an appropriate selection agent.

Referring again to FIG. 1, a bioreactor can include an inlet stream 14 that, in one embodiment, can be utilized to feed one or more extremozymes and/or extremophiles to the reaction chamber. In some embodiments, an inlet stream 14 can also be utilized to provide a material continuously or periodically to the reaction chamber. In other embodiments, a system can include multiple inlets which can independently be utilized to provide useful materials to a reaction chamber 10. A material fed to the reaction chamber 10 can be one that encourages an extreme environmental condition in the reaction chamber for decontamination purposes. For instance, an inlet 14 can be utilized to provide a salt to the reaction chamber either periodically or continuously in those embodiments in which the extremozyme is a halophilic enzyme. By way of example, the salt content of the reaction chamber 10 can be maintained during a reaction period at a concentration of, e.g., about 1 M or greater, about 1.5 M or greater, or about 2 M or greater in some embodiments. Any suitable salt an be utilized in such an embodiment, including, without limitation, an alkali meta or alkaline earth metal salt such as sodium chloride, potassium chloride, calcium chloride, magnesium chloride, sodium bromide, potassium bromide, sodium iodide, potassium iodide, or mixtures thereof, to maintain a desired salt content in the reaction chamber 10.

When considering an acidophilic or alkalophilic embodiment, an inlet 14 can be utilized to provide a suitable acid or base to the reaction chamber. For instance, in an embodiment in which an extremozyme of a process is acidophilic an acid can be provided to the reaction chamber 10 via an inlet 14 in order that the pH of within the reaction chamber 10 can be maintained during a reaction period at a pH of, e.g., about 5.5 or less, for instance about 5 or less, about 4 or less, about 3 or less, or about 2 or less in some embodiments. Acids as may be fed to a reaction chamber 10, for instance via inlet 14, can include strong or weak acids, and inorganic or organic acids, with preferred selection generally depending upon the specific environmental conditions desired for a reaction procedure.

In an embodiment in which an extremozyme of a process is alkaliphilic, a base can be provided to the reaction chamber 10 via an inlet 14 in order that the pH of within the reaction chamber 10 can be maintained during a reaction period at a pH of, e.g., about 7.5 or higher, for instance about 8 or higher, about 9 or higher, or about 10 or higher in some embodiments. Bases as may be fed to a reaction chamber 10, for instance via inlet 14, can include strong or weak bases, and inorganic or organic bases, with preferred selection generally depending upon the specific environmental conditions desired for a reaction procedure.

Other environmental control elements can be utilized to modify/control an environmental condition in the reaction chamber 10. For instance, in an embodiment in which an extremozyme of a process is a thermophile or a cryophile, a reaction chamber 10 can include heating elements or cooling elements, for instance can be jacketed with water or steam jackets (not shown in FIG. 1) to maintain desired temperature in the reaction chamber 10. By way of example, a temperature in the reaction chamber 10 can be maintained at a temperature of about 40° C. or greater, about 45° C. or greater, about 50° C. or greater, or about 55° C. or greater for a reaction period in thermophilic embodiments, for instance from about 40° C. to about 80° C., or from about 45° C. to about 75° C. in some thermophilic embodiments. Similarly, a temperature in the reaction chamber 10 can be maintained at a temperature of about 10° C. or lower, about 7° C. or lower, about 0° C. or lower, or about −10° C. or lower for a reaction period in cryophilic embodiments, for instance from about −20° C. to about 0° C., or from about −15° C. to about −5° C. in some thermophilic embodiments.

In those embodiments in which an extremozyme of a process is a piezophilic enzyme, a reaction chamber 10 can be in communication with a compressor 11, for instance via a high pressure line 15. In other embodiments, the reaction chamber can be in communication with a suitable high pressure gas source, for instance when the reaction procedure desirable occurs at high or low oxygen content, or in the presence of a particular gaseous compound, e.g., carbon dioxide or the like. In a high pressure embodiment, the reaction chamber 10 can be suitably isolated from the surrounding atmosphere such that the reaction chamber can be maintained at a high pressure for a period of time during which the contents of the reaction chamber 10 can be decontaminated and a biopolymer within the reaction chamber 10 can be degraded by an extremozyme. Byway of example, a pressure within the reaction chamber 10 can be maintained at about 10 kPa or greater, about 100 kPa or greater, about 500 kPa or greater, about 1 MPa or greater, about 10 MPa or greater or about 50 MPa or greater in some embodiments.

In some embodiments, a decontamination process can incorporate the use of an ionizing radiation, for instance in those embodiments in which an extremozyme exhibits radiation resistance. In such an embodiment, a reaction chamber can include a radiation source that can deliver suitably high energy radiation, e.g., high energy ultra-violet radiation, gamma rays, X-rays, etc. of about 1000 Gy or greater, to the contents of the reaction chamber 10.

Other compounds and processes that can enhance the sensitivity of pathogens to a decontamination process and/or that can enhance the degradation of a biopolymer without negatively impacting the other component of the process can be incorporated in a reaction system. For instance, the addition of a gas such as CO₂, the application of ultrasonic energy to the reaction chamber 10, inclusion of low levels of denaturants (e.g., urea, SDS, sodium sulfite, guanidine) to the reaction chamber 10, addition of trans-cinnamaldehyde, or the addition solubilizers/emulsifiers (e.g., polysorbates, propanediol, cetearyl glucoside, cetearyl alcohol, Tamasolve® (1-butylpyrrolidin-2-one; CAS 3470-98-2) to the reaction chamber 10 can enhance a procedure.

One or more extremozymes, and/or extremophiles that express an extremozyme can be located in a reaction chamber 10 in any suitable fashion that will encourage contact between the extremozyme(s) and a biopolymer. For instance, in one embodiment, a reaction chamber 10 can include a bed 13 that can include a polymer to be processed and enzyme and/or enzyme expressing cells adsorbed onto or otherwise contained within the bed 13. An enzyme and/or enzyme expressing cell can be pre-loaded onto a bed 13, can be periodically or continuously fed to the reaction chamber 10; e.g., via an inlet 14, or some combination thereof.

A second inlet 16 can provide continuous or periodic feed of a polymer to a reaction chamber 10 for simultaneous degradation and decontamination. Any PHA polymer can be degraded and decontaminated according to the present disclosure. A PHA can be a homopolymer or a copolymer. In one embodiment, a PHB-containing material can be fed to the reaction chamber 10.

Examples of monomer units that can be incorporated in PHA for processing as described can include 2-hydroxybutyrate, glycolic acid, 3-hydroxybutyrate, 3-hydroxypropionate, 3-hydroxyvalerate, 3-hydroxyhexanoate, 3-hydroxyheptanoate, 3-hydroxyoctanoate, 3-hydroxynonanoate, 3-hydroxydecanoate, 3-hydroxydodecanoate, 4-hydroxybutyrate, 4-hydroxyvalerate, 5-hydroxyvalerate, and 6-hydroxyhexanoate. Examples of PHA homopolymers include poly 3-hydroxyalkanoates (e.g., poly 3-hydroxypropionate (PHP), poly 3-hydroxybutyrate (PHB), poly 3-hydroxyvalerate (PHV), poly 3-hydroxyhexonoate (PHH), poly 3-hydroxyoctanoate (PHO), poly 3-hydroxydecanoate (PHD), and poly 3-hydroxy-5-phenylvalerate (PHPV)), poly 4-hydroxyalkanoates (e.g., poly 4-hydroxybutyrate (hereinafter referred to as PHB) and poly 4-hydroxyvalerate (hereinafter referred to as PHV)), or poly 5-hydroxyalkanoates (e.g., poly 5-hydroxyvalerate (hereinafter referred to as PHV)).

In certain embodiments, the PHA can be a copolymer (containing two or more different monomer units) in which the different monomers are randomly distributed in the polymer chain. Examples of PHA copolymers include poly 3-hydroxybutyrate-co-3-hydroxypropionate (hereinafter referred to as PHB3HP), poly 3-hydroxybutyrate-co-4-hydroxybutyrate (hereinafter referred to as P3HB4HB), poly 3-hydroxybutyrate-co-4-hydroxyvalerate (hereinafter referred to as PHB4HV), poly 3-hydroxybutyrate-co-3-hydroxyvalerate (hereinafter referred to as PHB3HV), poly 3-hydroxybutyrate-co-3-hydroxyhexanoate (hereinafter referred to as PH33HH) and poly 3-hydroxybutyrate-co-5-hydroxyvalerate (hereinafter referred to as PHB5HV).

In one embodiment, the post-consumer product(s) fed to the reaction chamber 10 via inlet 16 can be preprocessed, for instance, chopped, ground; etc. to provide a large surface area for interaction between biopolymer of the product(s) and enzyme within the reaction chamber 10. In embodiments in which decontamination of the post-consumer product(s) include high temperature, chilling the polymer-containing materials to be processed before elevating the temperature for the enzymatic process can enhance the thermal inactivation of pathogenic microbial cells. Inlet 16 can feed particulate matter including post-consumer PHA through the inlet 16, e.g., a screw type feeder, which can be positioned at the side of the reactor generally near the top of the reaction zone 12. e.g., above the bed 13.

An inlet 14 can optionally provide flow through the bed 13 to encourage reaction between extremozyme and biopolymer. In some embodiments, inlet 14 can provide a flow upward through the reaction chamber 10 and inlet 16 can provide continuous or periodic flow of polymer into the reaction chamber 10.

In one embodiment, inlet 14 can be near the bottom of a reaction chamber 10 and can provide a continuous flow upward through the bed 13 during the reaction period. As the flow from inlet 14 moves upward through the reaction chamber 10, enzyme any other components (e.g.; salts, acids, bases) can likewise move upward through the bed 13 and after degrading PHA in the lower regions of the bed can contact non-degraded polymer at the upper end of the bed 13. Simultaneously, environmental conditions within the reaction chamber 10 can decontaminate the polymer and any other components associated with the polymer in the post-consumer product(s). As such, it may be beneficial in some embodiments to provide materials to the reaction zone 10 via the upward flow from inlet 14. During the ongoing degradation process, volume of polymer initially fed to the bed 13 can degrade and additional polymer can be added to the reactor at the top of the bed 13. Thus, enzyme can contact the newly fed polymer, and the rate of addition of polymer can be roughly equal to the rate of enzymatic hydrolysis.

The reaction chamber can also include an outlet 18 above the bed 13 through which the degraded and decontaminated polymer and any other remaining components of the process can exit the reactor 10. Flow through the reactor can be controlled such that the retention time within the bed provides contact between the enzyme and the polymer suitable for hydrolysis reaction. The top of the bed 13 can be fitted in one embodiment with a plate to prevent remaining polymer particles and/or solid waste to exit via outlet 18.

Following exit via outlet 18, the reaction product flow can pass through a separator 20, within which any escaped polymer particulate and/or enzyme can be separated from the reactor outflow. For instance, in one embodiment, enzyme can be retained in the reaction zone 12 by immobilization on a support such as a polymeric bead, gel, etc. and the separator 20 can include a physical separation operation to remove any such support material from the outflow and return it to the reactor via line 22. Physical separation can also be utilized to separate any solid waste remaining following the simultaneous decontamination/degradation process from liquid waste.

A system can optionally include a separation operation 15 that can separate the product stream from the reactor into various products, e.g., PHA degradation products (re-usable monomers and/or oligomers) 17, decontaminated waste 19, other polymers 21, etc., for instance via a distillation separation or the like Beneficially, as the entire content of the reaction chamber 10 is decontaminated during a procedure, any waste from the procedure, either liquid or solid waste, can be simply and safely discarded.

FIG. 2 illustrates another embodiment of a disclosed system. In this embodiment, a reaction chamber 100 can be a component of a smaller system for instance as may be utilized for household waste, small business waste, a community or neighborhood waste treatment center, etc. As illustrated, a system can include a reaction chamber 100 into which one or more post-consumer products can be located following use. In a smaller design embodiment, a reaction chamber define an internal volume of, for example from about 10 L to about 500 L, for instance from about 15 L to about 300 L, from about 20 L to about 150 L, or from about 25 L to about 100 L, in some embodiments. An inlet 114 can be utilized in some embodiments to provide reactants or other useful materials to the reaction chamber 100. For instance, in one embodiment, inlet 114 can provide a temporary or permanent connection to a residential water tap for water flow into a reaction chamber.

Depending on the extremozyme(s) for use in the system and the desired decontamination approach, a system can include one or more components configured to attain a decontamination environmental condition within the reaction chamber 100. By way of example, in one embodiment, a system can include a chamber 110 that can carry a reactant, e.g., a salt; acid, base, or other useful material, for controlled release during a processing operation. For instance, a chamber 110 can hold a quantity of a salt and during a procedure, water can flow via inlet 114 to the chamber 110, upon which an amount of the salt can be dissolved and carried by the water to the reaction chamber 110. Similarly, a chamber 110 that contains an acid or base reactant can be designed to controllably release a Predetermined amount of the reactant into the reaction chamber 110 for use during a batch-type degradation/decontamination operation.

Alternatively, in those embodiments in which an environmental condition within the reaction chamber 110 is attained by use of an added material, the material (salt, base, acid) can be added to the reaction chamber 110 by a user, for instance in conjunction with the addition of an extremozyme or separately, as desired.

Other components can be utilized to control the environmental conditions within a reaction chamber including one or more of temperature control systems, e.g., heating or cooling coils 104 surrounding the reaction chamber 110, a high pressure source 115 in communication with a reaction chamber 110, and/or a high energy radiation source, e.g., a high energy UV light 112 within the reaction chamber 110.

In carrying out a decontamination/degradation procedure, a user can simply load the reaction chamber with a used product in conjunction with enzyme, environmental control materials, or other materials as discussed above that can encourage the procedure. A reaction chamber can then be sealed, for instance by a lid 120 that can optionally be locked during a procedure, Water can be fed to the reaction chamber 114 and the reaction chamber can optionally include a stirring device, e.g.; Paddles or the like, that can encourage contact between the post-consumer product(s) and the extremozyme(s) during the course of the simultaneous decontamination/degradation procedure.

Following a predetermined reaction period, which can generally be from a few minutes to several hours, depending upon the enzymes, the decontamination conditions, and the products to be treated, the contents of the reaction chamber can be emptied. For example, a reaction period can be about 5 minutes or longer, about 15 minutes or longer, about 30 minutes or longer, or about an hour or longer, such as from about 5 minutes to about 3 hours in some embodiments. As the contents of the reaction chamber 110 are decontaminated by the procedure, the post-treatment contents can be simply and safely discarded. For example, liquid waste and solid waste can be discarded together via a drain line 121 or optionally, solid waste can be removed separately, for instance by utilization of a disposable liner 125 that can be placed in the reaction chamber 100 prior to carrying out a procedure and removed following a procedure while the liquid waste can be drained via drain line 121.

The present disclosure may be better understood with reference to the Examples set forth below.

### Example 1

Six PHBDase enzymes were isolated from extremophiles, cloned into protein expression vectors, and purified for examination. The enzymes included two isolated thermophiles, Lihuaxuella thermophila and Schlegelella sp. ID0723, two isolated from halophiles, Halomonas aquamarine and Halomarina oriensis, an alkalophilic enzyme isolated from Alkalimonas amylolytica, and an acidophilic enzyme isolated from Acidiphilium cryptum. The two thermophilic enzymes depolymerize PHB at elevated temperatures; greater than temperatures at which enteric bacteria can live. The halophilic enzymes perform the same reaction in the presence of salt; with an optima of the Halomonas aquamarine enzyme at approximately 1 M NaCl and the Halomarina oriensis enzyme able to function at salt concentrations beyond 1 M; both at a higher concentrations than enteric bacteria can survive. In addition, a PHB deploymerase was isolated from the mesophile Pseudomonas geniculata to serve as a control/comparison. The origin of the specific enzyme sequences used for expression is shown in Table 2.

TABLE 2

| | Enzyme sequence |
|---|---|
| Bacterium | Accession number |
| Lihuaxuella thermophila | WP_089972404 |
| Schlegelella sp. ID0723 | WP_176065299 |
| Pseudomonas geniculata | RTY01006 |
| Halomarina oriensis | WP_158204529 |
| Halomonas aquamarina | WP_089674669 |
| Alkalimonas amylolytica | WP-091344878 |
| Acidiphilium cryptum | WP-050751056 |

All enzymes were identified as PHBDase, both by sequence homology as well as by enzymatic activity of the purified protein. The general sequence characteristics are shown in Table 3.

TABLE 3

| | | | Isoelectric | Number of |
|---|---|---|---|---|
| | # Amino | MW | Point | cysteines |
| Bacterium | acids* | (kDa) | (pl) | (# Cys) |
| Lihuaxuella thermophila | 301 | 32.3 | 5.39 | 2 |
| Schlegelella sp. ID0723 | 316 | 33.7 | 8.79 | 6 |
| Pseudomonas geniculata | 308 | 32.8 | 5.51 | 4 |
| Halomarina oriensis | 460 | 47.4 | 4.04 | 4 |
| Halomonas aquamarina | 331 | 35.5 | 4.39 | 8 |
| Alkalimonas amylolytica | 625 | 65.4 | 4.25 | 14 |
| Acidiphilium cryptum | 357 | 38.4 | 11.6 | 4 |

*after removal of any signal sequence and including the N-ter glycine

### PHB Depolymerase Expression Constructs

The amino acid sequence of the Lihuaxuella thermophila, Schlegelella sp. ID0723, Pseudomonas geniculata, Halomarina oriensis, Halomonas aquamarine, Acidiphilium cryptum, and Alkalimonas amylolytica PHD enzymes were utilized to construct a recombinant DNA expression system. First, the identified signal sequence was removed from some of the enzyme sequences (the first 22 amino acids for the L. thermophilia homolog, the first 30 amino acids from the H. halomarina homolog, the first 27 amino acids from the P. geniculata homolog, and the first 24 amino acids from the H. oriensis homolog. For the Acidiphilium cryptum sequence, the entire 357 amino acid protein was used as there is no signal sequence in this enzyme. Similarly, for the Alkalimonas amylolytica enzyme, the entire 625 amino acid protein was used as there is no signal sequence in this enzyme.

A histidine expression sequence and a TEV protease cleavage signal sequence: MHHHHHHGSENLYFQG (SEQ ID NO: 1) were appended to the amino terminal portion of each enzyme sequence. Upon cleavage the recombinant proteins will have an N-ter sequence that begins with a glycine residue. This new amino acid sequence was reverse translated to DNA and codon optimized for expression in E. coli using the program Gene Designer from ATUM, Inc. The gene was assembled using standard PCR techniques by ATUM, Inc. and cloned into the expression vector p454-MR (ampr, medium strength ribosomal binding site). The insert was verified by DNA sequencing after construction.

### Expression and Purification of the Enzymes

Each of the expression plasmids was used to transform chemically competent Oragami2-(DE3) bacteria. Single colonies were selected from LB-Amp plates and used for expression screening. Colonies were grown at 37° C. for 12 hours in LB media supplemented with 100 □g/mL ampicillin. This culture was used to inoculate fresh LB-AMP flasks at a 1:100 inoculum. These cultures were grown at 37° C. until OD595=0.4 (typically 4 hours) at which time IPTG was added to a final concentration of 1 mM. Growth was continued for 12 hours. Cells were harvested by centrifugation at 10,000×g for 15 minutes and frozen at −80° C. until use (minimal time frozen was 24 hours). Cells were thawed on ice and were resuspended in Buffer A (0.5 M NaCl, 20 mM Tris-HCl, 5 mM imidazole, pH 7.9) (typically 1 mL per gram of cells). Cells were disrupted via two passes through a French Press followed by centrifugation at 30,000×g for 30 minutes. The crude extract was mixed with an equal volume of charged His-Bind resin slurry and the mixture was poured into 5 cm×4.9 cc column. The column was washed with 10 column volumes of was buffer (0.5 M NaCl, 20 mM Tris-HCl, 60 mM imidazole, pH 7.9) at a flow rate of 0.2 mL/min. Enzyme was eluted from the column with the addition of 3 column volumes of 0.5 M NaCl, 20 mM Tris-HCl, 1.0 M imidazole, pH 7.9. Fractions were collected (1.0 mL). Fractions containing enzyme were pooled after analysis by SDS PAGE. The pooled fractions were applied to a 70 cm×4.9 cc Sephadex G-75 column (10 mM Tris-HCl, pH 7.5, 1 mM EDTA). Fractions containing homogeneous protein were pooled (after inspection by SDS PAGE), concentrated to 5 mg/mL via Centricon filters. Enzyme was stored frozen at −20° C. until use. The histidine tag region was removed from the enzymes using TEV protease. For the acidophile and alkaliphile samples, the enzymes were used with the N-ter histidine tag still in place. Protein was diluted to 1.0 mg/mL into 10 mM Tris-HCl, pH 7.5, 25 mM NaCl. 100 U of TEV protease was added per mg of enzyme (approximate ratio of 1:100 (w/w). The reaction was allowed to proceed for 16 h at 4° C. The mixture was passed over a charged nickel column. One column volume of eluent was collected representing purified tag-free enzyme.

### PHB Depolymerase Assay

An assay was utilized to measure β-hydroxybutyrate (HB) directly using the Sigma-Aldrich hydroxybutyrate assay kit MAK272. HB was measured fluorometrically (λex=535 nm, λem=587 nm). Aliquots (10 μL) were removed from the PHB depolymerase reaction at various time points, mixed with 50 μL of the supplied HB assay buffer, and pipetted into a well of a black, flat bottomed, 96-well plate. The plate was incubated at room temperature in the dark for 30 minutes. Fluorescence emission intensity was measured using a Molecular Dynamics SpectraMax M5. Fluorescence readings were converted to HB concentration via comparison to a standard curve constructed from known concentrations of pure hydroxybutyrate. All kinetic parameters are calculated per Segel (1993).

### Results

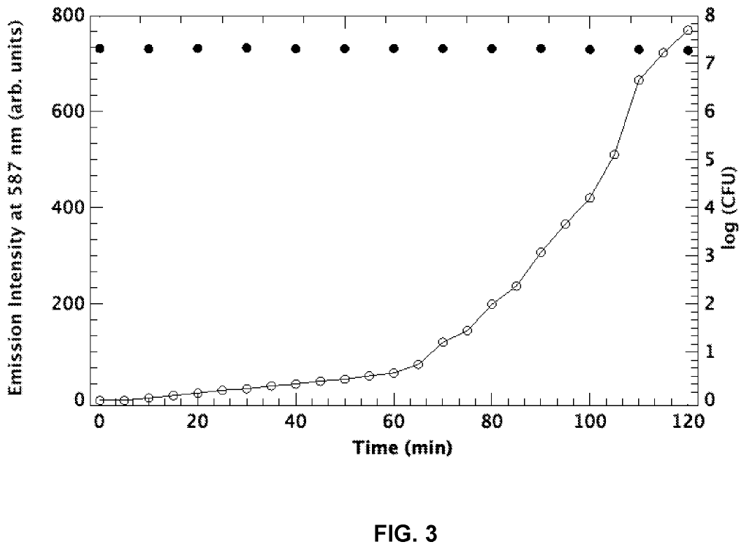

FIG. 3 presents enzymatic activity as a function of time in the fluorometric HB assay for the PHBDase from Pseudomonas geniculata (open circles) and also presents the colony forming units remaining as a function of time (closed circles). Reaction conditions included 10 mM Tris-HCl pH 7.0, 5 mM KCl, 5 mM MgCl², 37° C. As seen in FIG. 3, the Pseudomonas enzyme degraded PHB film in a dose dependent manner in the presence of added bacteria over a two hour reaction time course. Measuring bacterial viability over the reaction course showed very little change in the number of bacteria. This indicates that the reaction parameters of 37° C. and pH 7.0 had no measurable impact on bacterial survival. This was demonstrated by plotting the log of the colony forming units as a function of time after seeding the reaction with approximately 10⁷ bacteria from an overnight culture.

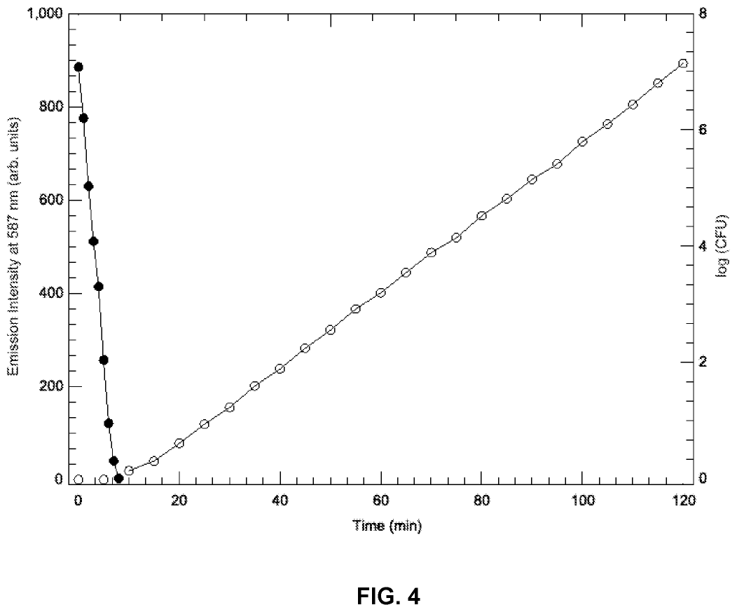

The Pseudomonas reaction served as a control showing that in the typical PHB depolymerase reaction environment, E. coli is fully viable. This contrasted greatly with the PHB depolymerase reaction catalyzed by the Lihuaxuella enzyme as illustrated in FIG. 4, which presents the PHB Depolymerase enzymatic activity as a function of time (open circles) and also presents the colony forming units remaining as a function of time (closed circles). Reaction conditions included 10 mM Na-acetate pH 6.0, 5 mM KCl, 5 mM MgCl₂, 55° C. The number of viable bacteria was reduced from 1.2×10⁷ cfu at time=0 to no viable bacteria measurable at t=8 minutes. Meanwhile the PHB film was depolymerized in a linear manner over the two hour time course after a short lag period.

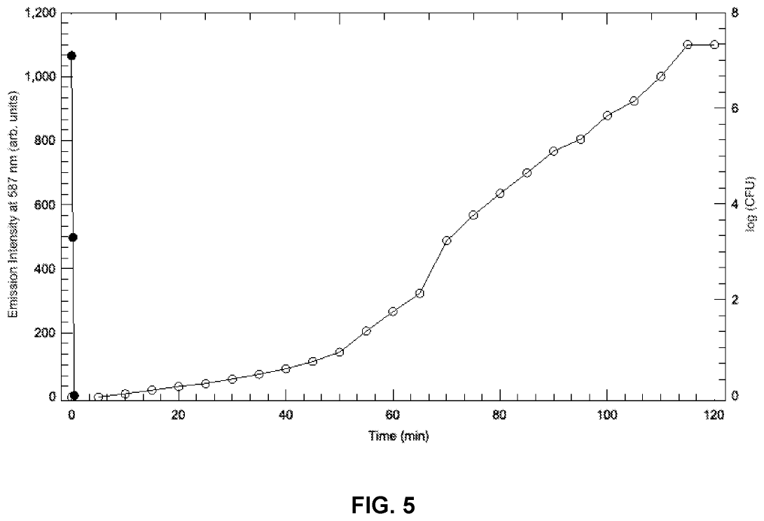

An even more striking temperature dependence is shown in FIG. 5, which illustrates the results when using the enzyme from Schlegelella. No viable bacteria were detected in the reaction after 30 seconds exposure to the high temperature environment (closed circles) (input bacteria=1.26×10⁷ cfu), but the PHB film is degraded within 100 minutes (open circles). Reaction conditions included 10 mM CHES pH 9.0, 5 mM KCl, 5 mM MgCl₂, 68° C. The Schlegelella enzyme had an optimal reaction temperature of 70° C.; significantly higher than the optimal viable temperature (or maximum viable temperature) of mesophilic bacteria.

The two halophilic enzymes were also analyzed. Halomonas aquamarina has an optimal salt concentration of approximately 1 M NaCl, whereas the archeon Halomarina oriensis is an extreme halophile with a salt optimum of 2.5 M, although it is found in environments up to 5 M NaCl.

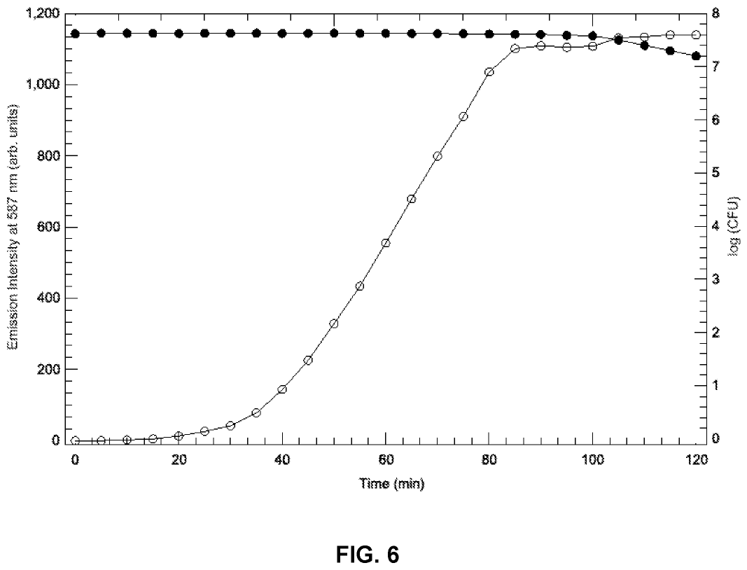

When the Halomonas enzyme was used to degrade a PHB film in the presence of 4.2×10⁷ input bacteria in a reaction environment that included the addition of 1 M NaCl, the reaction was completed in approximately 80 minutes (FIG. 6, open circles). As is shown in FIG. 6, the bacteria were relatively stable and viable over the time course, with only a slight decrease in viability after 100 minutes of exposure (final bacteria viability measurement at t=120 minutes was 1.6×10⁷ cfu) (FIG. 6, closed circles). Reaction conditions included 10 mM PIPES pH 6.5, 5 mM KCl, 1M NaCl, 5 mM MgCl₂, 37° C.

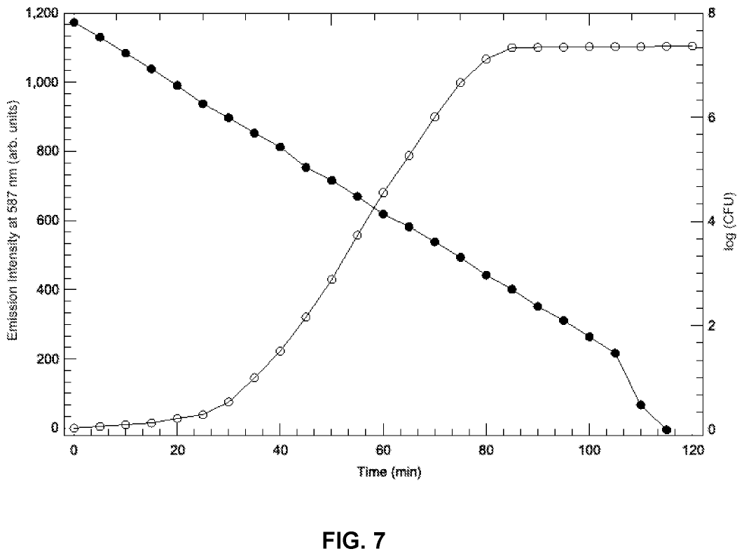

The E. coli were significantly less viable in the reaction catalyzed by the Halomarina enzyme in 2 M NaCl as is seen in FIG. 7. Viability was reduced from 6.6×10⁷ cfu at the start of the reaction to no detectable viable bacteria at the end of the time course (FIG. 7, closed circles). Meanwhile the archaeal enzyme fully degraded the PHB film within the first 80 minutes of the reaction (FIG. 7, open circles). Reaction conditions included 10 mM PIPES pH 6.5, 5 mM KCl, 2 M NaCl, 5 mM MgCl₂, 37° C.

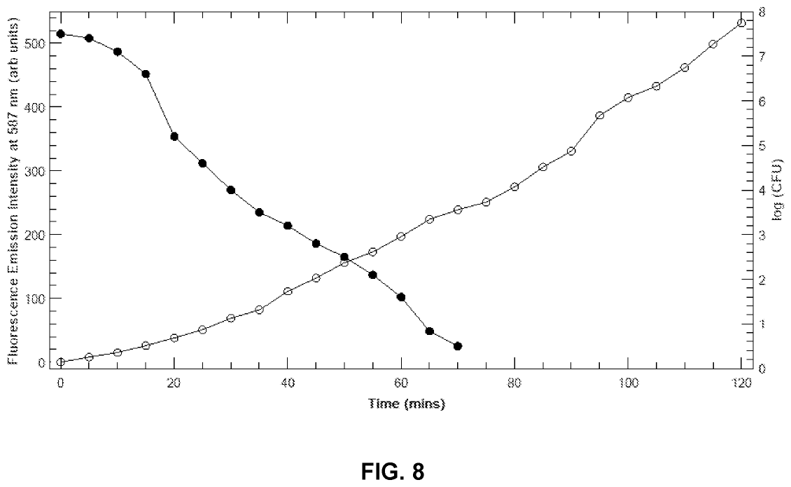

As shown in FIG. 8, the Acidiphilium cryptum enzyme was capable of depolymerizing PHB at pH 3.5 (open circles). At this pH the added E. coli survived for approximately 70 minutes during the reaction, with an approximate 4-log reduction in 40 minutes (closed circles). Reaction conditions included 10 mM Citric acid pH 3.5, 5 mM KCl, 5 mM MgCl₂, 37° C.

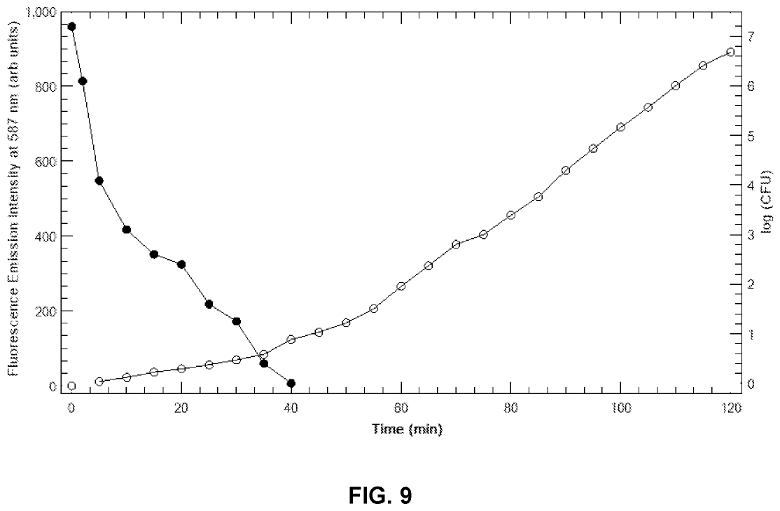

The Alkalimonas amylolytica enzyme was capable of depolymerizing PHB at pH 9.5 as is shown in FIG. 9 (open circles). At this pH the added E. coli survived for approximately 40 minutes during the reaction, with an approximate 4-log reduction in 20 minutes (closed circles). Reaction conditions included 10 mM CHES pH 9.5, 5 mM KCl, 5 mM MgCl₂, 37° C.

### Example 2

The amino acid sequence of the four PHBase enzymes from the organisms listed in Table 4, below, were obtained from Genbank, any signal sequences were identified and removed from the sequence, and a histidine expression sequence MHHHHHHGS (SEQ ID NO: 2) was added to the beginning (extreme N-ter). This new amino acid sequence (Table 5) was reverse translated to DNA and codon optimized for expression in E. coli using the program Gene Designer from ATUM, Inc. The gene was assembled using standard PCR techniques by ATUM, Inc. and cloned into the expression vector p454-MR (ampr, medium strength ribosomal binding site). The insert was verified by DNA sequencing after construction.

TABLE 4

| Organism | Accession number |
|---|---|
| Thermobifida halotolerans | WP_068692693 |
| Georgenia satyanarayanai | WP_146237554 |
| Natronococcus sp. LS1_42 | WP_148858780 |
| Marinobacter profundi | WP_099614009 |

TABLE 5

| | # Amino | MW | | |
|---|---|---|---|---|
| Bacterium | acids* | (kDa) | pl | # Cys |
| Thermobifida halotolerans | 378 | 40.2 | 5.29 | 8 |
| Georgenia satyanarayanai | 358 | 38.1 | 5.16 | 2 |
| Natronococcus sp. LS1_42 | 463 | 49.8 | 4.58 | 3 |
| Marinobacter profundi | 546 | 58.1 | 4.24 | 12 |

*after removal of any signal sequence and including the N-ter glycine

### Expression and Purification of the Enzymes

Each of the expression plasmids was used to transform chemically competent Oragami2-(DE3) bacteria. Single colonies were selected from LB-Amp plates and used for expression screening. Colonies were grown at 37° C. for 12 hours in LB media supplemented with 100 μg/mL ampicillin. This culture was used to inoculate fresh LB-AMP flasks at a 1:100 inoculum. These cultures were grown at 30° C. until OD595=0.4 (typically 4 hours) at which time IPTG was added to a final concentration of 1 mM. Growth was continued for 12 hours. Cells were harvested by centrifugation at 10,000×g for 15 minutes and frozen at −80° C. until use (minimal time frozen was 24 hours). Cells were thawed on ice and were resuspended in Buffer A (0.5 M NaCl, 20 mM Tris-HCl, 5 mM imidazole, pH 7.9) (typically 1 mL per gram of cells). Cells were disrupted via two passes through a French Press followed by centrifugation at 30,000×g for 30 minutes. The crude extract was mixed with an equal volume of charged His-Bind resin slurry and the mixture was poured into 5 cm×4.9 cc column. The column was washed with 10 column volumes of wash buffer (0.5 M NaCl, 20 mM Tris-HCl, 60 mM imidazole, pH 7.9) at a flow rate of 0.2 mL/min. Enzyme was eluted from the column with the addition of 3 column volumes of 0.5 M NaCl, 20 mM Tris-HCl, 1.0 M imidazole, pH 7.9. Fractions were collected (1.0 mL). Fractions containing enzyme were pooled after analysis by SDS PAGE. The pooled fractions were applied to a 70 cm×4.9 cc Sephadex G-75 column (10 mM Tris-HCl, pH 7.5, 1 mM EDTA). Fractions containing homogeneous protein were pooled (after inspection by SDS PAGE), concentrated to 5 mg/mL via Centricon filters. Enzyme was stored frozen at −20° C. until use. All proteins were assayed with the histidine tag in place.

### PHB Depolymerase Assay

An assay was utilized to measure β-hydroxybutyrate directly using the Sigma-Aldrich hydroxybutyrate assay kit MAK272. HB was measured fluorometrically (λex=535 nm, λem=587 nm). Aliquots (10 μL) were removed from the PHB depolymerase reaction at various time points, mixed with 50 μL of the supplied HB assay buffer, and pipetted into a well of a black, flat bottomed, 96-well plate. The plate was incubated at room temperature in the dark for 30 minutes. Fluorescence emission intensity was measured using a Molecular Dynamics SpectraMax M5. Fluorescence readings were converted to HB concentration via comparison to a standard curve constructed from known concentrations of pure hydroxybutyrate. All kinetic parameters are calculated per Segel (1993).

### Bacterial Growth

A standard laboratory stock of E. coli was used to inoculate 10.0 mL of Luria Broth (LB) and the culture was grown overnight at 37° C. with shaking at 300 rpm. This starting culture was used to inoculate 250 mL of LB at a 1:1000 ratio. The culture flask was incubated at 37° C. with shaking at 300 rpm for 12 hours. The bacteria were pelleted by centrifugation (5,000×g) for 10 minutes. The pellet was resuspended and washed in 1×PBS, recentrifuged, and washed again. The final pellet was resuspended in 1×PBS to a final concentration of 1×10⁹ cfu/mL and were kept on ice until used in the enzyme assay. Bacteria were used within a day of preparation. The PHB assays were spiked with 4×10⁷ cfu of the E. coli preparation and aliquots were removed at various time intervals (under the polyextreme reaction conditions as well as single extreme reaction conditions as controls), serially diluted in 1×PBS and plated on LB agar plates. After incubation at 37° C. for 12 hours colonies were counted. Survivability under single or multiple extreme conditions was plotted as log (cfu) vs. time in minutes.

### Results

None of the enzymes of this example have not been characterized in the literature but are clearly identifiable as bona fide PHBases from the existence of the PHBase consensus structures in the primary sequence. The enzyme selection covered the polyextreme conditions of high temperature, high pH, high pressure, and/or high salt and allowed for testing fecal bacterial survivability under these diverse reaction conditions.

All of the enzymes could be overexpressed and purified to homogeneity or near homogeneity. No effort was made to optimize either the expression conditions or the purification procedures. Typical yields were approximately 15.0 mg/L, which is typical of other PHBases previously studied by the authors. The enzyme from the benthic organism M. profundi was expressed at 15° C. to maximize protein stability. Unlike all the other enzymes, this PHBase could not be refrozen and was kept on ice until used. It was also notably unstable and lost all activity after 1-2 weeks. The reaction conditions used in this work are summarized in Table 6. No attempt was made to individually optimize pH or other buffer conditions as all the enzymes were active within the standard buffer component part of the reaction mix. The depolymerization of PHB could be demonstrated under all these conditions. No attempt was made in this study to determine the kinetic parameters (Kₘ, k_{cat}, Vₘₐₓ) for each enzyme, but the amount of enzyme added to the assay was standardized to 2.0 mg total in the reaction volume.

TABLE 6

| | Temp | [salt] | | Pressure |
|---|---|---|---|---|
| Organism | (° C.) | (M) | pH | (MPa) |
| Thermobifida halotolerans | 45 | 1.5 | 9.0 | 0.1 |
| Georgenia satyanarayanai | 40 | 1.0 | 10.0 | 0.1 |
| Natronococcus sp. LS1_42 | 50 | 2.0 | 10.0 | 0.1 |
| Marinobacter profundi | 4 | 1.5 | 9.0 | 150 |

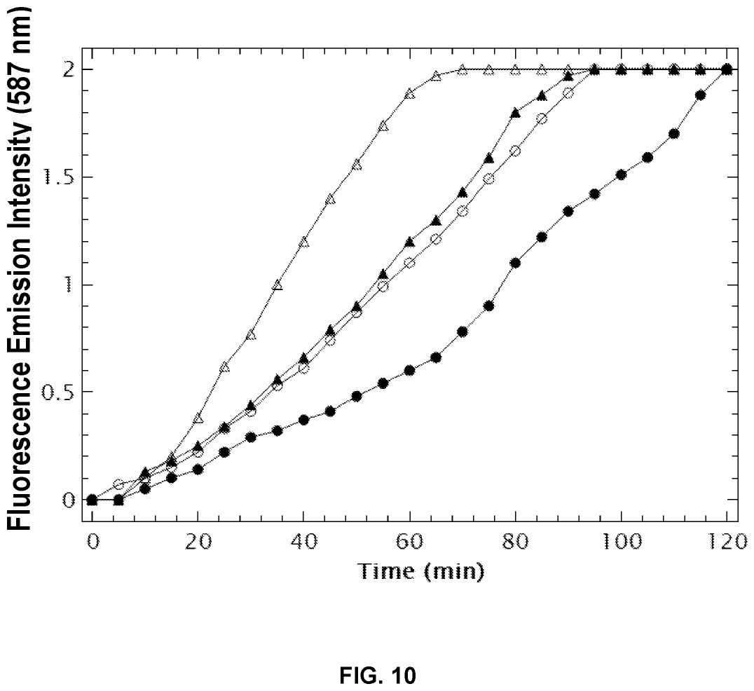

All four enzymes were capable of depolymerizing PHB as measured by the release of hydroxybutyrate in the enzyme assay under their specific reaction conditions (Table 6). The presence of 4×10⁷ cfu E. coli did not impair the reaction. This is important for an industrial process that would take a contaminated PHB-based consumer product and degrade it. For this to work well in practice, the contaminating bacteria cannot interfere with enzymatic activity. The measurement of released hydroxybutyrate from the polymer for the four enzymes is shown in FIG. 10. In FIG. 10, open triangles—Thermobifida halotolerans; closed triangles—Natronococcus sp. LS1_42; Open circles—Georgenia satyanarayanai; and closed circles—Marinobacter profundi.

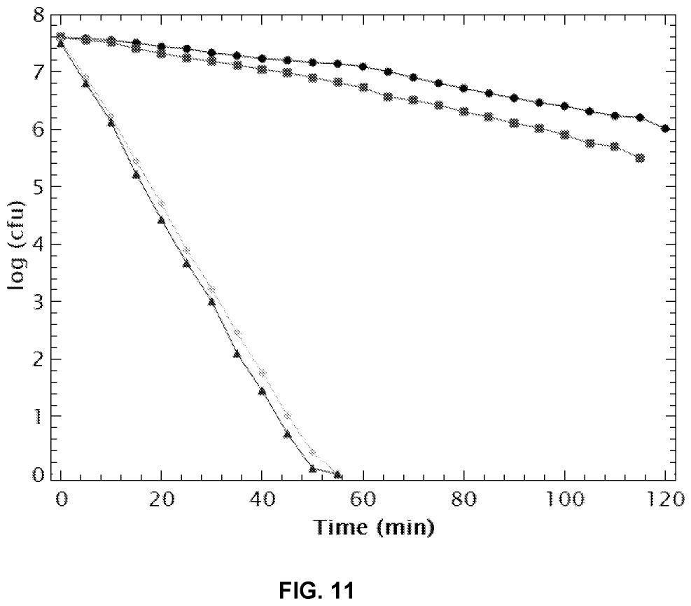

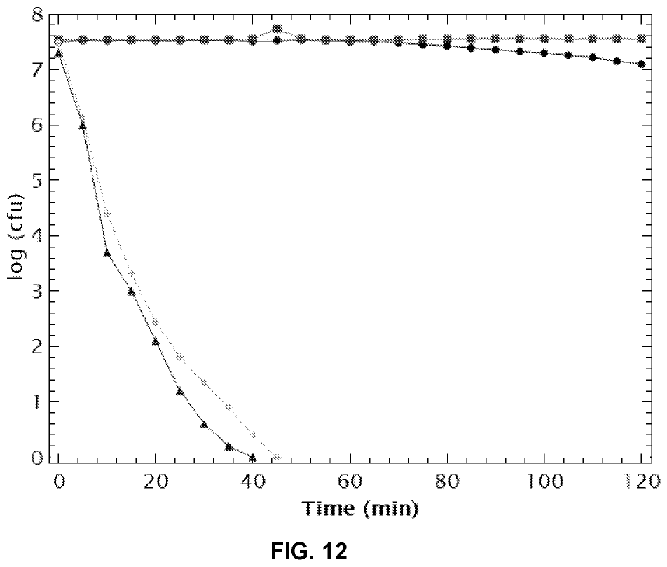

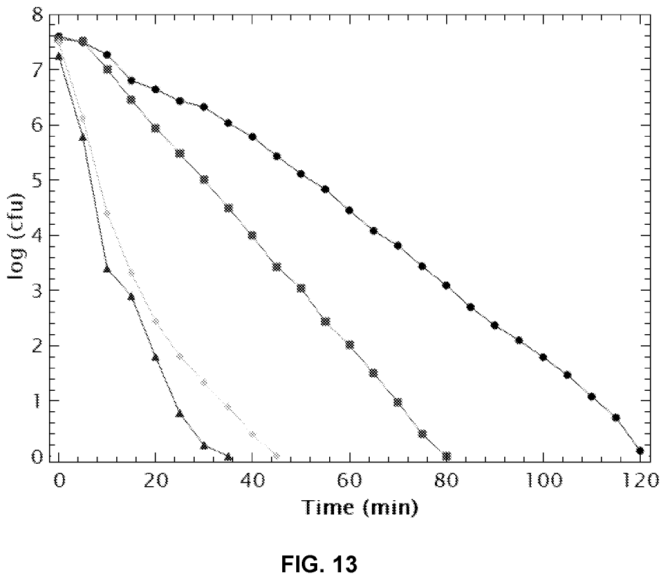

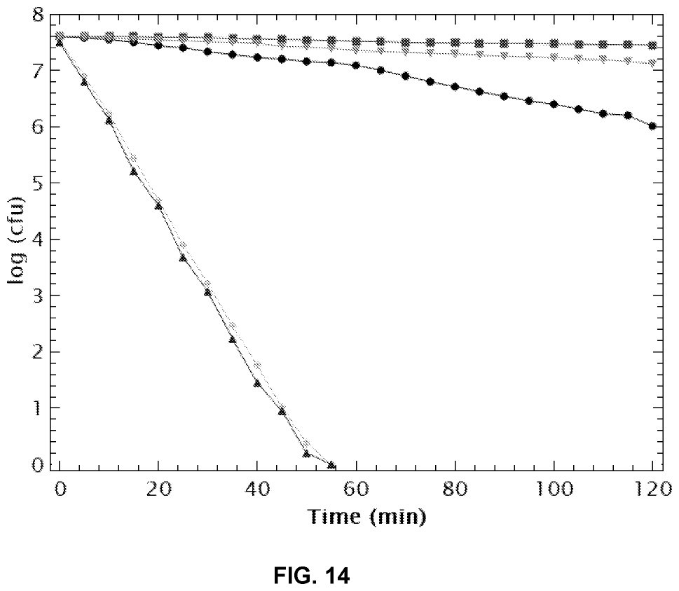

The survivability of 4×10⁷ cfu E. coli in the four reaction conditions was determined and compared to bacterial survival in each extreme condition by itself. The four survival plots are shown in FIG. 11, FIG. 12, FIG. 13, and FIG. 14 for reaction conditions from Table 6 of Thermobifida halotolerans, Georgenia satyanarayanai, Natronococcus sp. LS1_42, and Marinobacter profundi PHBase, respectively. As can be seen in the plots and in the calculated half lives in Table 7, there was not a synergistic increase in the rate of E. coli death under polyextreme conditions. The rate of bacterial death was driven primarily by the most deleterious single condition. That is, the half-life of E. coli in the combined polyextreme reaction conditions for all four enzyme environments was nearly identical (or somewhat simply additive) to the single condition that killed bacteria most efficiently, whether that is temperature or pH. Salt concentration had a lesser effect as did temperatures that are closer to the mesophile ideal. Table 7-10, below, details the conditions illustrated in each of FIG. 11-14, respectively.

TABLE 7
(FIG. 11)

| | [NaCl] | pH | Temp (° C.) |
|---|---|---|---|
| Circle | 1.5M | 7 | 30 |
| Diamond | 150 mM | 9 | 30 |
| Square | 150 mM | 7 | 45 |
| Triangle | 1.5M | 9 | 45 |

TABLE 8
(FIG. 12)

| | [NaCl] | pH | Temp (° C.) |
|---|---|---|---|
| Circle | 1.0M | 7 | 30 |
| Diamond | 150 mM | 10 | 30 |
| Square | 150 mM | 7 | 40 |
| Triangle | 1.0M | 10 | 40 |

TABLE 9
(FIG. 13)

| | [NaCl] | pH | Temp (° C.) |
|---|---|---|---|
| Circle | 2.0M | 7 | 30 |
| Diamond | 150 mM | 10 | 30 |
| Square | 150 mM | 7 | 50 |
| Triangle | 2.0M | 10 | 50 |

TABLE 10
(FIG. 14)

| | [NaCl] | pH | Temp (° C.) | Pressure (MPa) |
|---|---|---|---|---|
| Circle | 1.5M | 7 | 30 | 0.1 |
| Diamond | 150 mM | 9 | 30 | 0.1 |
| Square | 150 mM | 7 | 4 | 0.1 |
| Inverted Triangle | 150 mM | 7 | 30 | 150 |
| Triangle | 1.5M | 9 | 4 | 150 |

Table 11, below, summarizes the survivability of E. coli under the various reaction conditions examined.

TABLE 11

| | |
|---|---|
| Condition | Half-life (min) |

| | | |
|---|---|---|
| 4° | C. | — |
| 40° | C. | — |
| 45° | C. | 41.9 |
| 50° | C. | 19.6 |

| | |
|---|---|
| 1.0M NaCl | — |
| 1.5M NaCl | — |
| 2.0M NaCl | 69.8 |

| | | |
|---|---|---|
| pH | 9.0 | 28.8 |
| pH | 10.0 | 11.7 |
| 150 | MPa | — |

| | |
|---|---|
| 45° C., 1.5M NaCl, pH 9.0 | 23.0 |
| 40° C., 1.0M NaCl, pH 10.0 | 9.4 |
| 50° C., 2.0M NaCl, pH 10.0 | 10.2 |
| 4° C., 1.5M NaCl, pH 9.0, 150 MPa | 26.8 |

While certain embodiments of the disclosed subject matter have been described using specific terms, such description is for illustrative purposes only, and it is to be understood that changes and variations may be made without departing from the spirit or scope of the subject matter.

1. A method for treatment of a post-consumer product, the method comprising:
within a reaction chamber, contacting a post-consumer product with an extremophilic polyhydroxyalkanoate depolymerase (PHADase) and/or a microorganism that expresses the extremophilic PHADase, the post-consumer product comprising a polyhydroxyalkanoate, the contact taking place at an environmental condition that is deleterious to a mesophilic pathogen and at which the PHADase is active, wherein upon the contact, the post-consumer product is decontaminated and the polyhydroxyalkanoate is degraded.

2. The method of claim 1, wherein the extremophilic PHADase comprises a polyextremophilic PHADase.

3. The method of claim 1, the method comprising contacting the post-consumer product with two or more extremophilic PHADase.

4-8. (canceled)

9. A system configured for simultaneous decontamination and biodegradation of a post-consumer product, the system comprising a reaction chamber configured to retain a post-consumer product that includes a polyhydroxyalkanoate in contact with an extremophilic polyhydroxyalkanoate depolymerase (PHADase) and/or an extremophile that expresses the extremophilic PHADase, the system further comprising an environmental control system configured to maintain an environmental condition within the reaction chamber that is deleterious to a mesophilic pathogen and at which the PHADase is active.

10. The system of claim 9, wherein the environmental condition that is deleterious to a mesophilic pathogen comprises one or more of a salt concentration of about 1.5 M or greater, a temperature of either about 40° C. or greater or about 10° C. or lower, a pH of either about 5.5 or less or about 7.5 or higher, a pressure of about 110 kPa or greater, and ionizing radiation of about 1000 Gy or greater.

11. The system of claim 9, further comprising a solid/liquid separator.

12-14. (canceled)

15. A cell that has been transformed to express an extremophilic PHADase that catalyzes degradation of a polyhydroxyalkanoate at an environmental condition that is deleterious to a mesophilic pathogen.

16. The cell of claim 15, wherein the cell is a yeast cell, an insect cell, a plant cell, a mammalian cell, or a bacterial cell.
